(19) United States
(12) Patent Application Publication
(10) Pub. No.: US 2025/0017595 A1
(43) Pub. Date: Jan. 16, 2025

(54) ANTI-MIGRATION STENT

(71) Applicant: BCM Co., LTD., Gyeonggi-do (KR)

(72) Inventor: Byung Cheol MYUNG, Gyeonggi-do (KR)

(21) Appl. No.: 18/496,929

(22) Filed: Oct. 30, 2023

(30) Foreign Application Priority Data
Jul. 11, 2023 (KR) 10-2023-0089800

(51) Int. Cl. A61B 17/12; A61B 17/00

(52) U.S. Cl. CPC A61B 17/12177; A61B 17/12109; A61B 17/12172; A61B 2017/00853; A61B 2017/00867; A61B 2017/00982

(57) ABSTRACT

Proposed is an anti-migration stent which expands a lesion area occurring in a lumen and is prevented from migrating, is deformed to fit the lumen of a curved shape, and can be easily removed from the lumen. More specifically, the anti-migration stent includes a first stent including a first cylindrical body, a first film part, and a second film part, and a second stent including a second cylindrical body, a connection part formed by having a diameter decreasing at one side of the second cylindrical body, and a third film part made of silicone formed on the second cylindrical body and the connection part, wherein after the second stent is fitted over one side of the first stent, the one side of the first stent and the connection part are connected to each other, and the second stent is held in the lumen.

### CROSS REFERENCE TO RELATED APPLICATION

The present application claims priority to Korean Patent Application No. 10-2023-0089800, filed on Jul. 11, 2023, the entire contents of which are incorporated herein for all purposes by this reference.

### BACKGROUND OF THE INVENTION

### Field of the Invention

The present disclosure relates to an anti-migration stent that expands a lesion area narrowed or blocked in the lumen in the body and is prevented from migrating in the lesion area when an external force occurs due to moving body fluids, pressure of food, or shaking of the human body.

### Description of the Related Art

Generally, when a lesion area that is narrowed or blocked in the lumen of the body, such as the esophagus, duodenum, or biliary tract occurs, the inherent function of the lumen of the body that moves body fluids and food is deteriorated. Accordingly, a stent is inserted into the lesion area that is narrowed or blocked to expand the narrowed lumen of the body.

In this regard, Patent Document 1 proposed a stent for biliary tract having a space part formed by weaving one or more strands of shape memory alloy wires together or crossing the wires in a zigzag manner and a cylindrical body with multiple bent ends formed along a circumference thereof at opposite ends thereof, with the entirety of the space part or cylindrical body being covered with silicone or PTFE harmless to the human body for movement of bile, wherein the stent includes a bile outlet having a diameter deceasing by a moving bent portion while a medical thread, in a zigzag manner, passes sequentially through the bent ends formed at the ends of the stent for biliary tract, and end space parts formed together therewith, and opposite ends of the medical thread are held and tied by being pulled to constrict the bent ends inward, and only a portion of a space part of the moving bent portion is coated with silicone or PTFE to block the portion of the space part.

However, in the case of the stent disclosed in Patent Document 1 described above, there was a risk of the stent migrating in the lesion area when external forces were generated due to body fluids moving along the lumen, pressure of food, or shaking of the human body.

### DOCUMENT OF RELATED ART

(Patent Document 1) Korean Patent No. 10-1996524

### SUMMARY OF THE INVENTION

Accordingly, the present disclosure has been made keeping in mind the above problems occurring in the related art, and the present disclosure is intended to propose an anti-migration stent that expands a lesion area narrowed or blocked in the lumen in the body; is prevented from migrating in the lesion area when an external force occurs due to moving body fluids, pressure of food, or shaking of the human body; is modified to fit the curved lumen of the body; and is configured to have a structure different from the structure of a conventional stent that the anti-migration stent can be easily removed from the lumen in the body when removed after a predetermined period of time after completing a procedure on the lesion area.

In order to achieve the above objectives, according to one aspect of the present disclosure, there is provided an anti-migration stent having a first stent configured to expand a lesion area narrowed or blocked in a lumen of a body and to have a first cylindrical body formed in a hollow cylindrical shape by weaving or crossing wires made of a super elastic shape memory alloy in a mesh shape so that multiple first space parts are formed between the wires, a first film part made of polytetrafluoroethylene (PTFE) formed on an inner surface of the first cylindrical body, with the first film part formed in a spiral shape to have predetermined intervals defined therein, and a second film part made of polytetrafluoroethylene (PTFE) formed on an entire outer surface of the first cylindrical body so that the second film part is bonded to the first film part, the anti-migration stent including: a second stent having multiple second space parts formed by weaving or crossing the wires made of a super elastic shape memory alloy in a mesh shape, with the second stent being shorter in length than the first stent and fitted over and connected to one side of the first stent from the outside so that the second stent is held in the lumen in which the lesion area has occurred, wherein the second stent is composed of a second cylindrical body formed in a hollow cylindrical shape, and a connection part formed by having a diameter decreasing at one side of the second cylindrical body so as to be connected to the first stent, with the second cylindrical body and the connection part having a third film part made of silicone formed thereon.

According to the present disclosure, the first stent expands a lesion area, and the second stent protruding from the first stent is held in the lumen, thereby preventing the migrating of the anti-migration stent in the lesion area during the occurrence of external forces due to moving body fluids, pressure of food, or shaking of the human body.

According to the present disclosure, since the third film part made of silicone rather than PTFE is formed on the second stent, the third film part made of silicone is thinner than a film part made of PTFE, and because of this, the second stent is easily turned over by the third film part formed to be thinner of silicone rather than PTFE when the first stent is pulled during removal of the first stent after a predetermined period of time elapses after the procedure of the lesion area, thereby enabling the anti-migration stent to be easily removed from the lumen of the body.

According to the present disclosure, the first film part made of PTFE is formed in a spiral shape on the inner surface of the first cylindrical body, and the second film part made of PTFE bonded to the first film part is formed on the entire outer surface of the first cylindrical body, and accordingly, various parts of the first stent at which the first and second film parts bonded to each other are not located are flexible.

That is, the anti-migration stent is flexibly transformed to fit the curved lumen and includes the first and second film parts made of PTFE that maintain a bonded state, thereby maintaining a curvedly transformed state.

In addition, since the first and second film parts are bonded in a long spiral shape along the longitudinal direction of the first cylindrical body, the first and second film parts widely support the first cylindrical body so that the anti-migration stent can maintain a curved state thereof, and the second film part formed on the entire outer surface of the first cylindrical body prevents the growth of the lesion area into the first stent through each of the first space parts.

According to the present disclosure, the first film part made of PTFE is formed on the entire inner surface of the first cylindrical body, and the second film part made of PTFE bonded to the first film part is formed in a spiral shape on the outer surface of the first cylindrical body, and thus various parts of the first stent at which the first and second film parts bonded to each other are not located are flexible. Because of this, the anti-migration stent is flexibly transformed to fit the curved lumen.

In addition, due to the first and second film parts made of PTFE maintaining a bonded state, the anti-migration stent maintains a curvedly transformed state, and since the first and second film parts are bonded in a long spiral shape along the longitudinal direction of the first cylindrical body, the first and second film parts widely support the first cylindrical body so that the anti-migration stent can maintain a curved state.

In addition, the lesion area is inserted into and held in the first space part which is not closed by the first and second film parts.

In addition, the first film part formed on the entire inner surface of the first cylindrical body prevents the growth of the lesion area into the first stent through the first space part.

According to the present disclosure, the first and second film parts made of PTFE bonded to each other on the inner and outer surfaces of the first cylindrical body are formed in the same spiral shapes, thereby forming the first and second film parts less on the first stent, and various parts of the first stent at which the bonded first and second film parts are not located are more flexible, thereby deforming the anti-migration stent of the present disclosure more flexibly to fit the more curved lumen.

In addition, due to the first and second film parts made of PTFE maintaining a bonded state, the anti-migration stent maintains a curvedly deformed state.

In addition, the first and second film parts are bonded in a long spiral shape along the longitudinal direction of the first cylindrical body, thereby supporting the cylindrical body widely so that the anti-migration stent can maintain a curved state.

In addition, the lesion area is inserted into and held in the first space part which is not closed by the first and second film parts.

According to the present disclosure, since a protruding part has no first and second film parts formed thereon, the lesion area and the lumen are inserted into and held in the first space part of the protruding part, thereby allowing the opposite sides of the anti-migration stent of the present disclosure to be held in the lumen by the second stent and the protruding part.

According to the present disclosure, since the connection part of the second stent is formed integrally with one end of the first stent, there is no need to connect the first and second stents to each other with a connecting thread, thereby facilitating manufacturing of the anti-migration stent of the present disclosure.

In addition, since no connecting thread is used, when the second stent is turned over, the connection part is prevented from being folded by a connecting thread, and thus the connection portion of the connection part is prevented from being turned over and exposed to the outside of the first stent, thereby reducing the holding of the connection part of the second stent to the lumen during removal of the anti-migration stent.

According to the present disclosure, since the first and second film parts are not formed on one side of the first cylindrical body overlapping with the connection part, the one side of the first cylindrical body is flexible, and accordingly, as the one side of the first cylindrical body is stretched by an external force pulling the first stent, the one side of the first cylindrical body has reduced volume thereof, that is, the anti-migration stent is less held in the lumen and is easily removed.

In addition, a connection area between one end of the first stent and the connection part is thin without first and second film parts formed on the connection area, and thus the connecting thread protrudes less to the outside of the second stent, thereby reducing the holding of the connecting thread and the second stent in the external tube of the stent delivery system when mounting the anti-migration stent on a stent delivery system.

In other words, the anti-migration stent can be easily mounted to the stent delivery system, and has no first and second film parts formed thereon, thereby simplifying the work of sewing the first and second space parts of the first and second stents with the connecting thread.

According to the present disclosure, since the connection part has no third film part formed thereon, the connection part is flexible. This allows the connection part of the second stent to be easily deformed and easily turned over by an external force pulling the first stent, thereby facilitating the removal of the anti-migration stent of the present disclosure.

In addition, a connection area between one end of the first stent and the connection part is thin without a third film part, and thus the connecting thread protrudes less to the outside of the second stent, thereby reducing the holding of the connecting thread and the second stent in the external tube of the stent delivery system when the anti-migration stent of the present disclosure is mounted on the stent delivery system.

In other words, the anti-migration stent can be easily mounted on the stent delivery system.

In addition, no third film part is formed, thereby simplifying the sewing of the first and second space parts of the first and second stents with the connecting thread.

According to the present disclosure, since first and second film parts are not formed on one side of the first cylindrical body overlapping with the connection part and a third film part is not formed on the connection part, the one side of the first cylindrical body and the connection part are flexible, and accordingly, as the one side of the first cylindrical body is stretched by an external force pulling the first stent, the one side of the first cylindrical body has reduced volume thereof.

In other words, the anti-migration stent is less held in the lumen and can be easily removed.

In addition, the connection part of the second stent is easily deformed and turned over by an external force pulling the first stent, thereby facilitating the removal of the anti-migration stent at a position at which the procedure of the anti-migration stent is performed on the lumen.

In addition, a connection area between one end of the first stent and the connection part is thin without first, second, and third film parts formed on the connection area and the connecting thread protrudes less to the outside of the second stent, thereby reducing the holding of the connecting thread and the second stent in the external tube of the stent delivery system when mounting the anti-migration stent on the stent delivery system.

In other words, the anti-migration stent can be easily mounted on the stent delivery system.

In addition, since no first, second, and third film parts are formed, the work of sewing the first and second space parts of the first and second stents with the connecting thread is simplified.

According to the present disclosure, since a first space part at one side of the first cylindrical body overlapping with the second stent is greater in size than a first space part of a remaining part of the first cylindrical body which does not overlap with the second stent, elasticity of the one side of the first cylindrical body overlapping with the second stent decreases, thereby reducing the volume of the one side of the first cylindrical body stretched by an external force pulling the first stent.

In other words, the anti-migration stent is less held in the lumen and is easily removed from the lumen.

According to the present disclosure, since each of the second space parts is greater in size than the first space part, the second stent has low elasticity and is easily deformed and turned over by an external force pulling the first stent, thereby facilitating the removal of the anti-migration stent of the present disclosure.

According to the present disclosure, the first stent protrudes from the connection part by a predetermined length and is connected to the connection part with the connecting thread, and thus one end of the first stent and one end of the connection part are simultaneously prevented from being held in the lumen, thereby reducing stimulation to the lumen.

In addition, when mounting the anti-migration stent on the stent delivery system, the anti-migration stent is less held in the external tube.

### BRIEF DESCRIPTION OF THE DRAWINGS

The above and other objectives, features, and other advantages of the present disclosure will be more clearly understood from the following detailed description when taken in conjunction with the accompanying drawings, in which:

FIGS. 1 to 6 and an exploded view, a combined view, and partially enlarged cross-sectional views of an anti-migration stent according to a first embodiment of the present disclosure;

FIGS. 7 to 10 are usage state views of the anti-migration stent according to the first embodiment of the present disclosure;

FIGS. 11 and 12 are a front view and a usage state view of the anti-migration stent according to a first modified example of the first embodiment of the present disclosure;

FIGS. 13 to 15 are a front view, a partially enlarged cross-sectional view, and a usage state view of the anti-migration stent according to a second modified example of the first embodiment of the present disclosure;

FIGS. 16 to 18 are a front view, a partially enlarged cross-sectional view, and a usage state view of the anti-migration stent according to a third modified example of the first embodiment of the present disclosure;

FIGS. 19 to 21 are a front view, a partially enlarged cross-sectional view, and a usage state view of the anti-migration stent according to a fourth modified example of the first embodiment of the present disclosure;

FIGS. 22 to 25 are front views and a usage state view of the anti-migration stent according to a fifth modified example of the first embodiment of the present disclosure;

FIG. 26 is a front view of the anti-migration stent according to a sixth modified example of the first embodiment of the present disclosure;

FIGS. 27 to 32 are an exploded view, a combined view, and partially enlarged cross-sectional views of the anti-migration stent according to a second embodiment of the present disclosure;

FIGS. 33 to 37 are usage state views of the anti-migration stent according to the second embodiment of the present disclosure;

FIGS. 38 and 39 are a front view and a usage state view of the anti-migration stent according to a first modified example of the second embodiment of the present disclosure;

FIGS. 40 to 42 are a front view, a partially enlarged cross-sectional view, and a usage state view of the anti-migration stent according to a second modified example of the second embodiment of the present disclosure;

FIGS. 43 to 45 are a front view, a partially enlarged cross-sectional view, and a usage state view of the anti-migration stent according to a third modified example of the second embodiment of the present disclosure;

FIGS. 46 to 48 are a front view, a partially enlarged cross-sectional view, and a usage state view of the anti-migration stent according to a fourth modified example of the second embodiment of the present disclosure;

FIGS. 49 to 52 are front views and a usage state view of the anti-migration stent according to a fifth modified example of the second embodiment of the present disclosure;

FIG. 53 is a front view of the anti-migration stent according to a sixth modified example of the second embodiment of the present disclosure;

FIGS. 54 to 59 are an exploded view, a combined view, and partially enlarged cross-sectional views of the anti-migration stent according to a third embodiment of the present disclosure;

FIGS. 60 to 64 are usage state views of the anti-migration stent according to the third embodiment of the present disclosure;

FIGS. 65 and 66 are a front view and a usage state view of the anti-migration stent according to a first modified example of the third embodiment of the present disclosure;

FIGS. 67 to 69 are a front view, a partially enlarged cross-sectional view, and a usage state view of the anti-migration stent according to a second modified example of the third embodiment of the present disclosure;

FIGS. 70 to 72 are a front view, a partially enlarged cross-sectional view, and a usage state view of the anti-migration stent according to a third modified example of the third embodiment of the present disclosure;

FIGS. 73 to 75 are a front view, a partially enlarged cross-sectional view, and a usage state view of the anti-migration stent according to a fourth modified example of the third embodiment of the present disclosure;

FIGS. 76 to 79 are front views and a usage state view of the anti-migration stent according to a fifth modified example of the third embodiment of the present disclosure; and

FIG. 80 is a front view of the anti-migration stent according to a sixth modified example of the third embodiment of the present disclosure.

### DETAILED DESCRIPTION OF THE INVENTION

Accordingly, embodiments of the present disclosure as described above will be described in detail with reference to the accompanying drawings as follows.

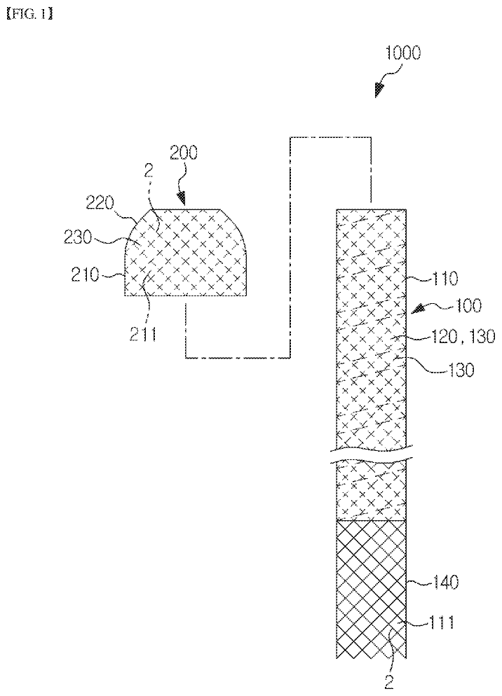

As illustrated in FIGS. 1 to 80, an anti-migration stent 1000 according to various embodiments and various modified examples of the present disclosure is inserted into the lumen 1 of the body such as the esophagus, duodenum, and biliary tract of the human body through the stent delivery system such as a catheter so as to expand a lesion area 1a narrowed and blocked in the lumen 1.

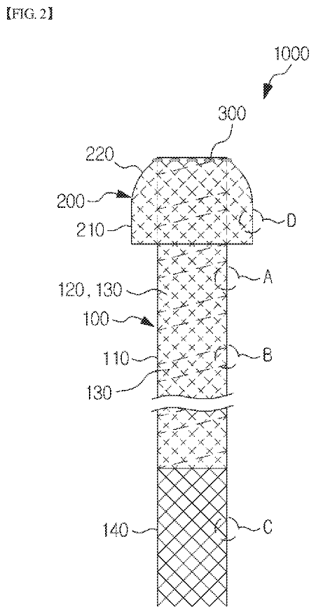

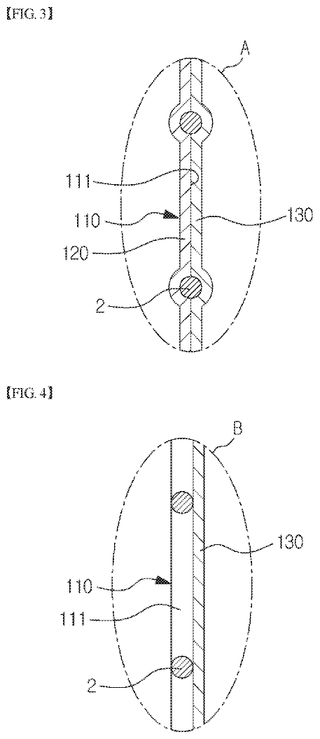

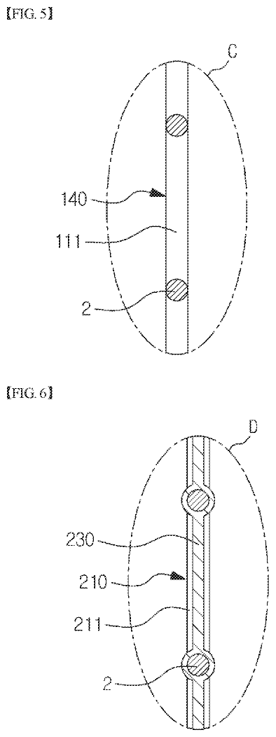

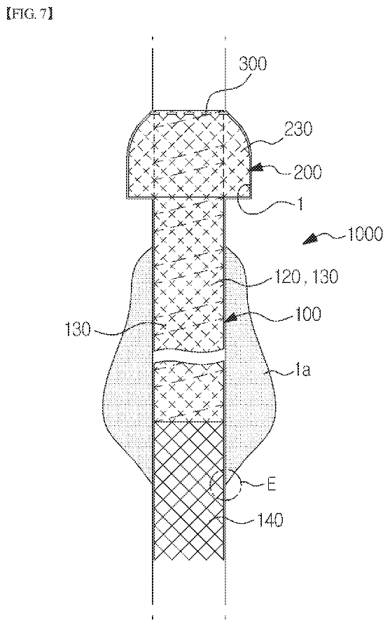

As illustrated in FIGS. 1 to 10, the anti-migration stent 1000 according to a first embodiment of the present disclosure includes: a first stent 100; and a second stent 200 fitted over one outer side of the first stent 100 to be connected thereto by a connecting thread 300.

The first stent 100 includes a first cylindrical body 110 formed in a hollow cylindrical shape by weaving and crossing wires 2 made of a super elastic shape memory alloy in a mesh shape so that multiple first space parts 111 are formed between the wires 2.

Here, the first stent 100 is heat-treated, and a weaving part in which wires 2 are woven and a crossing part in which the wires 2 cross are formed around each of the first space parts 111, and thus the first space part 111 is formed in a shape similar to a rhombus.

The first stent 100 includes: the first film part 120 made of polytetrafluoroethylene (PTFE) formed on the inner surface of the first cylindrical body 110, with the first film part 120 formed in a spiral shape to have predetermined intervals defined therein; and the second film part 130 made of polytetrafluoroethylene (PTFE) formed on the entire outer surface of the first cylindrical body 110, with the second film part 130 bonded to the first film part 120.

Here, the first and second film parts 120, 130 are bonded to each other by heating and pressing, and some of the multiple first space parts 111 are closed by the first and second film parts 120, 130 bonded to each other.

That is, the first space parts 111 closed by the first and second film parts 120, 130 are not easily deformed by an external force, and the first film part 120 is formed in a spiral shape on a portion of the inner surface of the first cylindrical body 110, so a portion of the second film part 130 formed on the entire outer surface of the first cylindrical body 110 is not bonded to the first film part 120.

Accordingly, a portion of the second film part 130 is free with no object to be bonded thereto, and thus the remaining first space parts 111 which are not closed by the first and second film parts 120, 130 may be deformed by an external force.

In other words, various parts of the first cylindrical body 110 are flexible due to the free second film part 130, and the remaining part of the first cylindrical body 110 is not flexible due to the first and second film parts 120, 130 maintaining a bonded state.

In addition, the intervals formed in the first film part 120 are regular or irregular.

The first stent 100 includes a protruding part 140 formed to protrude on one side of the first cylindrical body 110 located at a side opposite to the second stent 200. At this time, the protruding part 140 has no first and second film parts 120, 130 formed thereon and thus the first space part 111 of the protruding part 140 is not closed.

The second stent 200 includes: a second cylindrical body 210 formed in a hollow cylindrical shape by weaving or crossing wires 2 made of a super elastic shape memory alloy in a mesh shape so that multiple second space parts 211 are formed between the wires 2, wherein the second cylindrical body 210 is shorter in length than the first cylindrical body 110 and is greater in diameter than the first cylindrical body 110; and a connection part 220 formed by having a diameter decreasing at one side of the second cylindrical body 210.

Here, the second stent 200 is heat-treated, and a weaving part in which wires 2 are woven and a crossing part in which the wires 2 cross are formed around each of the second space parts 211, and thus the second space part 211 is formed in a shape similar to a rhombus. The second stent 200 includes a third film part 230 made of silicone formed on the second cylindrical body 210 and the connection part 220.

As for the third film part 230, multiple second space parts 211 are impregnated or spray-coated with a silicone solution, and each of the second space parts 211 is closed with the third film part 230 made of silicone.

To explain the reason for which the third film part 230, which is made of silicone rather than PTFE, is formed on the second stent 200, when the third film part is made of PTFE, one pair of third film parts is formed and is located to face each other on the inner and outer surfaces of the second stent 200, and then bonded to each other by heating and pressing, so the third film part made of PTFE thicker than the third film part 230 made of silicone is formed on the second stent 200.

Because of this, when the first stent 100 of the anti-migration stent 1000 is pulled to be removed when a predetermined period of time elapses after completing the procedure of the anti-migration stent 1000 on the lesion area 1a, the second stent 200 may be overlap in several layers by being rolled due to the thick PTFE film part. At the same time, the second stent 200 overlapped in several layers is rigid and thus may not be turned over in a direction opposite to the pulling direction of the first stent 100.

That is, when the anti-migration stent 1000 does not come out of the lumen 1, there is a risk that the lumen 1 may be damaged by the rigid second stent 200.

However, in this case, although there are the problems as described above, when the second stent 200 intended to prevent migrating moves downward when the anti-migration stent 1000 of the present disclosure is about to migrate, the second stent 200 is rolled up and overlapped in several layers and a radial force thereof becomes stronger. Accordingly, the second stent 200 rather has the effect of preventing the anti-migration stent 1000 from migrating.

However, although there is the advantage, there are ultimately the above-mentioned problems when removing the anti-migration stent 1000. Accordingly, in order to solve these problems, it is advantageous that the third film part 230 made of silicone rather than PTFE is formed on the second stent 200 of the present disclosure so that the third film part 230 is thin.

In other words, the third stent 200 of the present disclosure has the third film part 230 made of silicone rather than PTFE to make the third film part 230 thin.

The anti-migration stent 1000 is manufactured by sewing and connecting the first space part 111 of one end of the first stent 100 and the second space part 211 of the connection part 220 to each other along a circumferential direction with the connecting thread 300 after the second stent 200 is fitted over one side of the first stent 100.

Here, one end of the first stent 100 and one end of the connection part 220 are located almost on the same line.

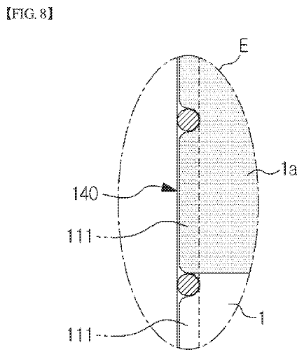

Accordingly, a procedure is performed by inserting the anti-migration stent 1000 into the lesion area 1a narrowed or blocked in the lumen 1 of the body, such as the esophagus, duodenum, and biliary tract of the human body, through a stent delivery system such as a catheter, and accordingly, as illustrated in FIG. 7, the first stent 100 expands the lesion area 1a occurring in the lumen 1 of the body, and the second stent 200 is inserted into and held in the lumen 1 in which the lesion area 1a has occurred.

In addition, the second film part 130 formed on the entire outer surface of the first cylindrical body 110 prevents the lesion area 1a from being inserted into the first space part 111. Of course, when the second stent 200 is located at the lesion area 1a, the third film part 230 prevents the lesion area 1a from being inserted into the second space part 211.

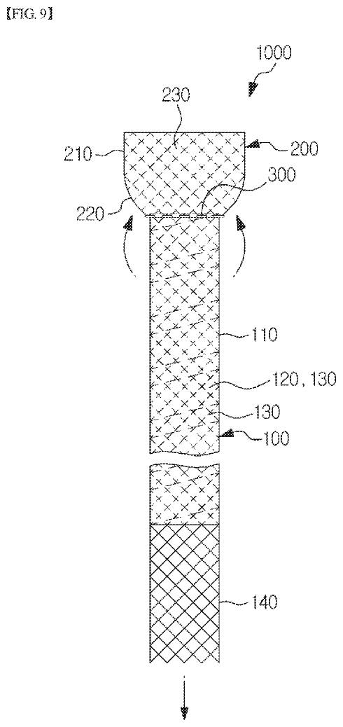

As illustrated in FIG. 8, the lumen 1 and the lesion area 1a are inserted into and held in the first space part 111 in the protruding part 140.

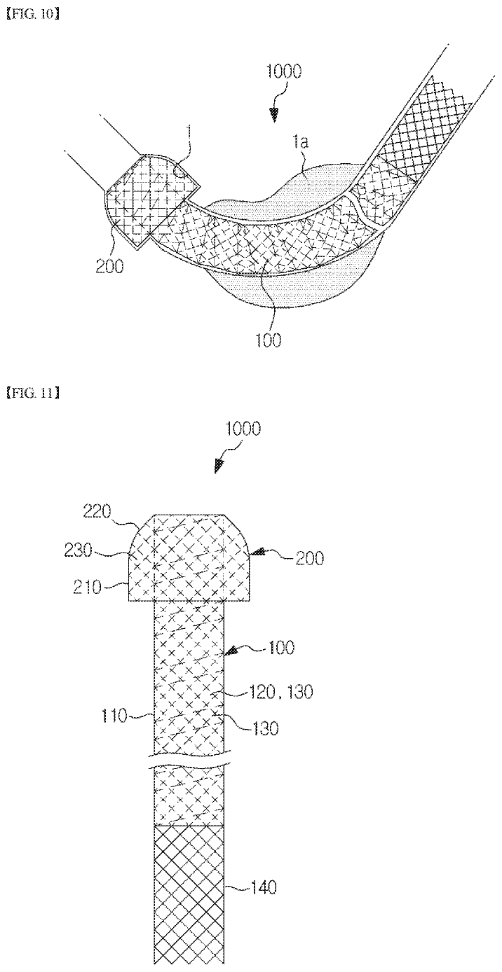

As illustrated in FIG. 9, when a predetermined period of time elapses after completing the procedure of the anti-migration stent 1000 on the lesion area 1a narrowed or blocked in the lumen 1 of the body, the anti-migration stent 1000 is required to be removed. In this case, the protruding part 140 of the first stent 100 of the anti-migration stent 1000 is pulled by a stent delivery system, such as a catheter.

Due to this action, the second stent 200 is turned over in a direction opposite to the pulling direction of the first stent 100 since the second stent 200 is in close contact with the lumen 1. Accordingly, the second stent 200 is not held in the lumen 1, and thus the anti-migration stent 1000 of the present disclosure can be easily removed from the lumen 1.

As illustrated in FIG. 10, when the anti-migration stent 1000 is located at the curved lumen 1, first space parts 111 are deformed in various parts of the first stent 100 flexible due to the free second film part 130, and this means that the anti-migration stent 1000 is easily deformed to fit the curved shape of the lumen 1.

In this case, the first space part 111 is not easily deformed in the remaining part of the first stent 100 which is not flexible due to the first and second film parts 120, 130 maintaining a bonded state. This means that unless the first space part 111 located at the free second film part 130 receives an external force, the first space part 111 maintains a deformed state due to the first space part 111 which is not easily deformed by being located at the first and second film parts 120, 130 maintaining a bonded state. That is, it means that the anti-migration stent 1000 maintains a curvedly deformed state.

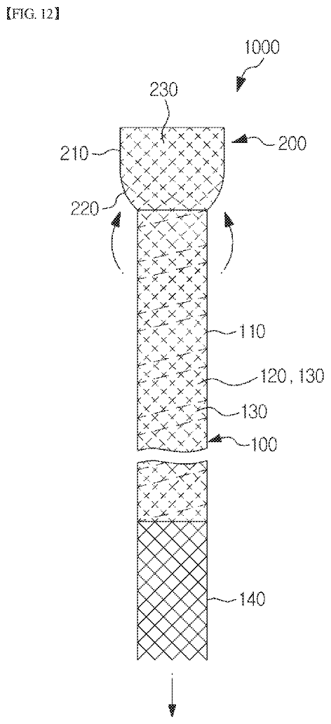

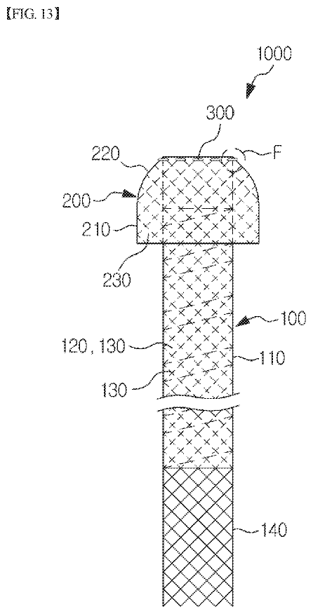

As illustrated in FIGS. 11 and 12, in the second stent 200 of the anti-migration stent 1000 according to the first modified example of the first embodiment of the present disclosure, without the connecting thread 300, the connection part 220 is integrally formed on and connected to one end of the first stent 100. When a predetermined period of time elapses after the procedure of the anti-migration stent 1000, there occurs a case in which the anti-migration stent 1000 is required to be removed. In this case, when the first stent 100 of the anti-migration stent 1000 is pulled, the second stent 200 is not held in the lumen 1 while being turned over in a direction opposite to the pulling direction of the first stent 100 since the second stent 200 is in close contact with the lumen 1, so the anti-migration stent 1000 is removed from the lumen 1.

In addition, since the connection part 220 does not have the connecting thread 300, the connection part 220 does not have a portion partially folded by a connecting thread 300.

That is, a connection portion of the connection part 220 connected to one end of the first stent 100 is prevented from protruding to the outer side of the first stent 100 while being turned over, so the anti-migration stent 1000 of the present disclosure is easily removed from the lumen.

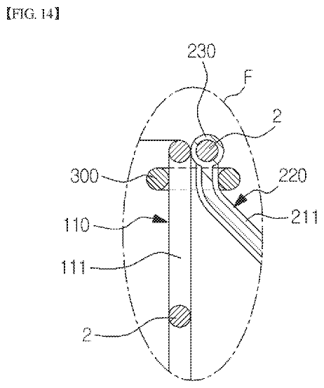

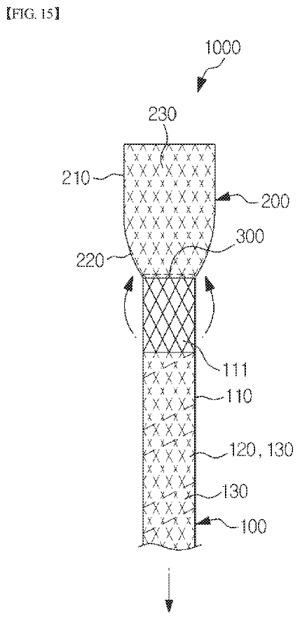

As illustrated in FIGS. 13 to 15, in the anti-migration stent 1000 according to the second modified example of the first embodiment of the present disclosure, one side of the first cylindrical body 110 overlapping with the connection part 220 has no first and second film parts 120, 130 formed thereon.

That is, the one side of the first cylindrical body 110 overlapping with the connection part 220 is flexible without first and second film parts 120, 130.

In addition, the connection portion of one end of the first stent 100 to the connection part 220 has no first and second film parts 120, 130 formed thereon and is configured to be thin.

According to such a configuration, when a predetermined period of time elapses after completing the procedure of the anti-migration stent 1000 on the lesion area 1a, the anti-migration stent 1000 is required to be removed. In this case, when the first stent 100 of the anti-migration stent 1000 is pulled, the second stent 200 is turned over in a direction opposite to the pulling direction of the first stent 100 since the second stent 200 is in close contact with the lumen 1, and one side of the first cylindrical body 110 flexible without the first and second film parts 120, 130 is easily deformed and stretched by an external force pulling the first stent 100, so the anti-migration stent 1000 of the present disclosure is easily removed from the lumen 1.

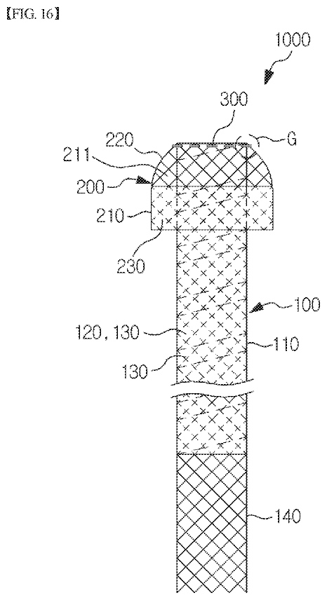

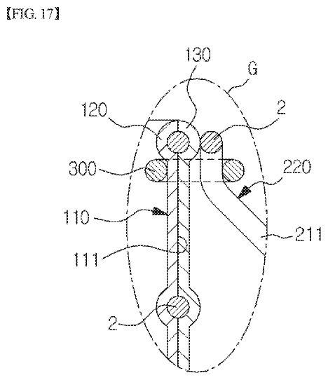

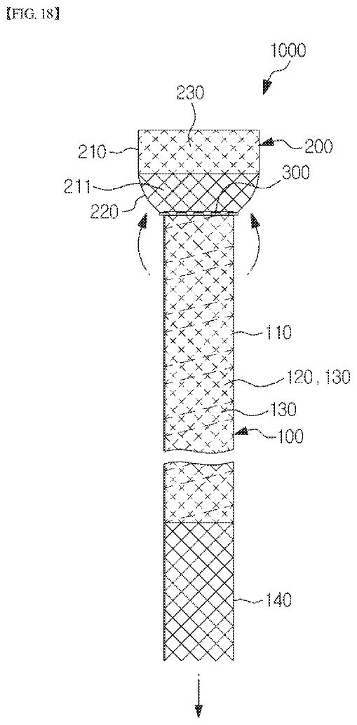

As illustrated in FIGS. 16 to 18, in the anti-migration stent 1000 according to the third modified example of the first embodiment of the present disclosure, the connection part 220 has no third film part 230 formed thereon.

That is, the connection part 220 is flexible without a third film part 230, and the connection portion of one end of the first stent 100 to the connection part 220 has no third film part 230 formed thereon and is configured to be thin.

According to such a configuration, when a predetermined period of time elapses after completing the procedure of the anti-migration stent 1000 on the lesion area 1a, the anti-migration stent 1000 is required to be removed. In this case, when the first stent 100 of the anti-migration stent 1000 is pulled, the second stent 200 is turned over in a direction opposite to the pulling direction the first stent 100 since the second stent 200 is in close contact with the lumen 1.

Here, due to the connection part 220 flexible without the third film part 230, the second stent 200 is easily deformed and turned over by an external force pulling the first stent 100.

For this reason, the anti-migration stent 1000 of the present disclosure is easily removed from the lumen 1.

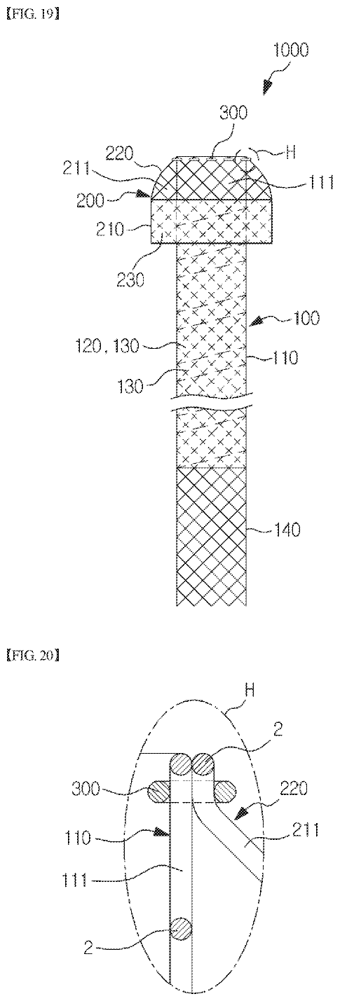

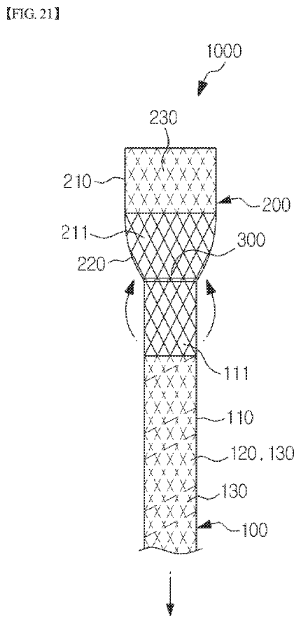

As illustrated in FIGS. 19 to 21, in the anti-migration stent 1000 according to the fourth modified example of the first embodiment of the present disclosure, one side of the first cylindrical body 110 overlapping with the connection part 220 has no first and second film parts 120, 130 formed thereon, and the connection part 220 has no third film part 230 formed thereon.

That is, the one side of the first cylindrical body 110 overlapping with the connection part 220 is flexible without the first and second film parts 120, 130, and the connection part 220 is flexible without a third film part 230.

In addition, the connection portion of one end of the first stent 100 to the connection part 220 has no first, second, and third film parts 120, 130, 230 formed thereon and is configured to be thin.

According to such a configuration, when a predetermined period of time elapses after completing the procedure of the anti-migration stent 1000 on the lesion area 1a, the anti-migration stent 1000 is required to be removed. In this case, when the first stent 100 of the anti-migration stent 1000 is pulled, the second stent 200 is turned over in a direction opposite to the pulling direction of the first stent 100 since the second stent 200 is in close contact with the lumen 1.

Here, due to the connection part 220 flexible without the third film part 230, the second stent 200 is easily deformed and turned over by an external force pulling the first stent 100.

In addition, one side of the first cylindrical body 110 flexible without first and second film parts 120, 130 is easily deformed and stretched by an external force pulling the first stent 100, so the anti-migration stent 1000 of the present disclosure is easily removed from the lumen 1.

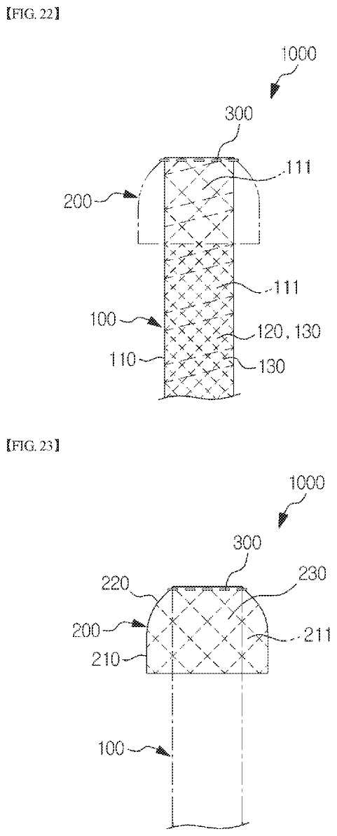

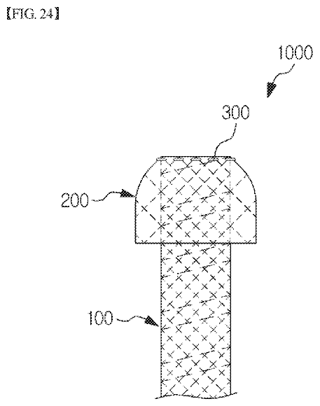

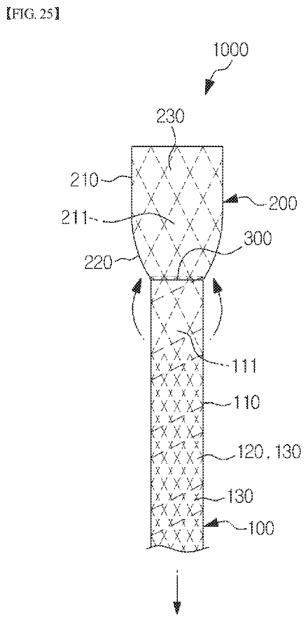

As illustrated in FIGS. 22 to 25, in the anti-migration stent 1000 according to the fifth modified example of the first embodiment of the present disclosure, the first space part 111 at one side of the first cylindrical body 110 overlapping with the second stent 200 is formed to be two to four times greater in size than a first space part 111 of a remaining part of the first cylindrical body 110 which does not overlap with the second stent 200.

That is, the one side of the first cylindrical body 110 overlapping with the second stent 200 has lower elasticity than the remaining part of the first cylindrical body 110 which does not overlap with the second stent 200.

According to such a configuration, when a predetermined period of time elapses after completing the procedure of the anti-migration stent 1000 on the lesion area 1a, the anti-migration stent 1000 is required to be removed. In this case, when the first stent 100 of the anti-migration stent 1000 is pulled, one side of the first cylindrical body 110 overlapping with the second stent 200, due to low elasticity thereof, is easily deformed and stretched by an external force pulling the first stent 100.

In addition, since the second space part 211 of the second stent 200 is formed to be two to four times greater in size than the first space part 111 of one side of the first cylindrical body 110 which does not overlap with the second stent 200, the elasticity of the second stent 200 decreases.

According to such a configuration, when a predetermined period of time elapses after completing the procedure of the anti-migration stent 1000 on the lesion area 1a, the anti-migration stent 1000 is required to be removed. In this case, when the first stent 100 of the anti-migration stent 1000 is pulled, the second stent 200 with low elasticity is easily deformed and easily turned over by an external force pulling the first stent 100, so the anti-migration stent 1000 of the present disclosure is easily removed from the lumen 1.

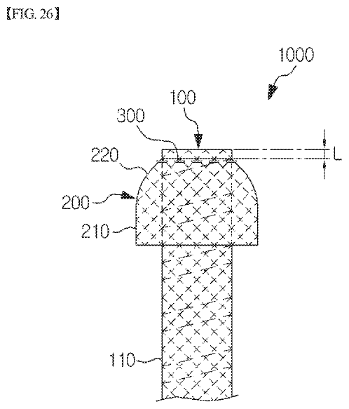

As illustrated in FIG. 26, the first stent 100 of the anti-migration stent 1000 according to the sixth modified example of the first embodiment of the present disclosure protrudes by a predetermined length L from the connection part 220 and is connected to the connection part 220 by sewing with the connecting thread 300. Here, since the length L is 3˜5 mm, one end of the first stent 100 and one end of the connection part 220 are not located on the same line.

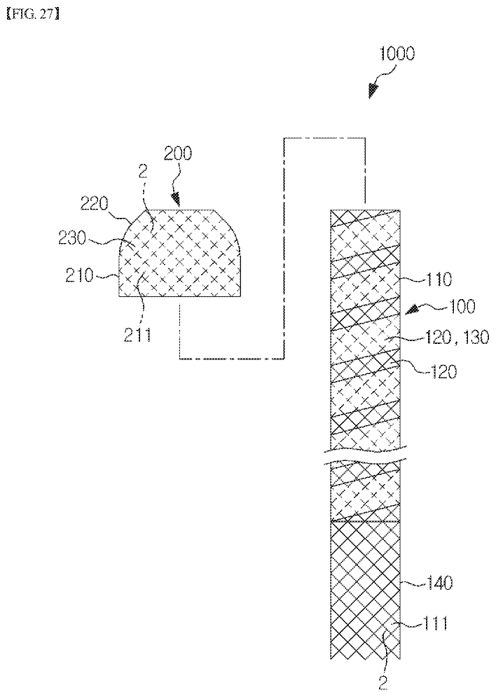

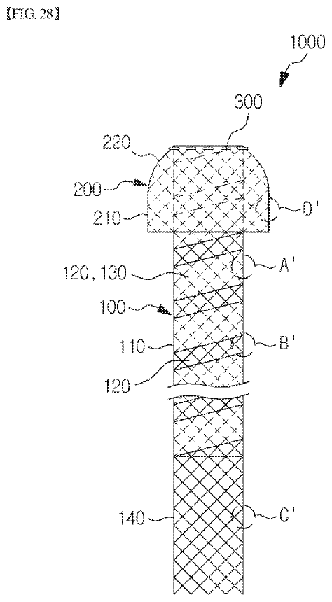

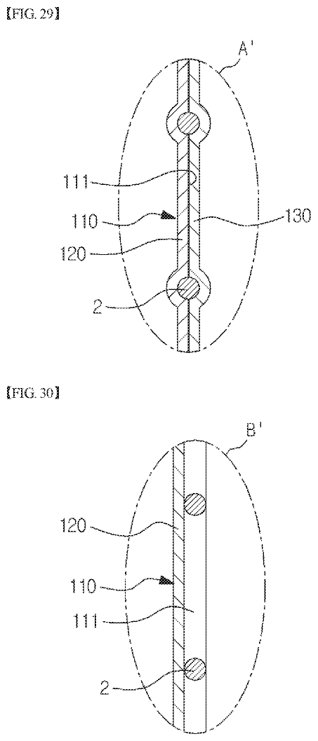

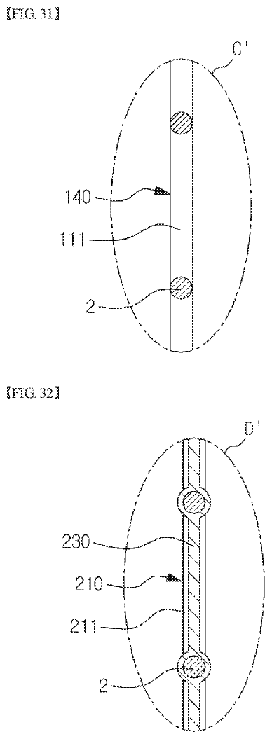

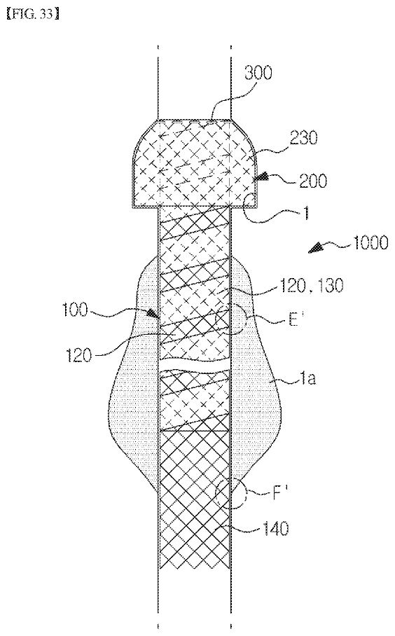

As illustrated in FIGS. 27 to 37, the anti-migration stent 1000 according to a second embodiment of the present disclosure includes: the first stent 100; the second stent 200 fitted over one outer side of the first stent 100 to be connected thereto by a connecting thread 300.

The first stent 100 includes the first cylindrical body 110 formed in a hollow cylindrical shape by weaving or crossing wires 2 made of a super elastic shape memory alloy in a mesh shape so that multiple first space parts 111 are formed between the wires 2.

Here, the first stent 100 is heat-treated, and a weaving part in which wires 2 are woven and a crossing part in which the wires 2 cross are formed around the first space part 111, and thus the first space part 111 is formed in a shape similar to a rhombus.

The first stent 100 includes the first film part 120 made of polytetrafluoroethylene (PTFE) formed on the entire inner surface of the first cylindrical body 110, and the second film part 130 made of polytetrafluoroethylene (PTFE) formed on the outer surface of the first cylindrical body 110, with the second film part 130 formed in a spiral shape to have predetermined intervals defined therein and bonded to the first film part 120.

Here, the first and second film parts 120 and 130 are bonded to each other by heating and pressing, and some of the multiple first space parts 111 are closed by the first and second film parts 120 and 130 bonded to each other.

That is, the first space part 111 closed by the first and second film parts 120, 130 is not easily deformed by an external force, and the second film part 130 is formed in a spiral shape on a portion of the outer surface of the first cylindrical body 110, so a portion of the first film part 120 formed on the entire inner surface of the first cylindrical body 110 is not bonded to the second film part 130. Accordingly, a portion of the first film part 120 is free with no object to be bonded thereto, and thus a remaining first space part 111 which is not closed by the first and second film parts 120, 130 may be deformed by an external force.

In other words, various parts of the first cylindrical body 110 is flexible due to the free first film part 120, and the remaining part of the first cylindrical body 110 is not flexible due to the first and second film parts 120, 130 maintaining a bonded state.

In addition, the intervals formed in the second film part 130 are regular or irregular.

The first stent 100 includes the protruding part 140 formed to protrude on one side of the first cylindrical body 110 located at a side opposite to the second stent 200. At this time, the protruding part 140 has no first and second film parts 120, 130 formed thereon, and thus the first space part 111 of the protruding part 140 is not closed.

The second stent 200 includes: the second cylindrical body 210 formed in a hollow cylindrical shape by weaving or crossing wires 2 made of a super elastic shape memory alloy in a mesh shape so that multiple second space parts 211 are formed between the wires 2, wherein the second cylindrical body 210 is shorter in length than the first cylindrical body 110 and is greater in diameter than the first cylindrical body 110; and the connection part 220 formed by having a diameter decreasing at one side of the second cylindrical body 210.

Here, the second stent 200 is heat-treated, and a weaving part in which wires 2 are woven and a crossing part in which the wires 2 cross are formed around the second space part 211, and thus the second space part 211 is formed in a shape similar to a rhombus. The second stent 200 includes the third film part 230 made of silicone formed on the second cylindrical body 210 and the connection part 220.

As for the third film part 230, multiple second space parts 211 are impregnated or spray-coated with a silicone solution, and each of the second space parts 211 is closed with the third film part 230 made of silicone.

To explain the reason for which the third film part 230, which is made of silicone rather than PTFE, is formed on the second stent 200, when the third film part is made of PTFE, one pair of third film parts is formed and is located to face each other on the inner and outer surfaces of the second stent 200, and then bonded to each other by heating and pressing, so the third film part made of PTFE thicker than the third film part 230 made of silicone is formed on the second stent 200.

Because of this, when the first stent 100 of the anti-migration stent 1000 is pulled to be removed when a predetermined period of time elapses after completing the procedure of the anti-migration stent 1000 on the lesion area 1a, the second stent 200 may be overlap in several layers by being rolled due to the thick PTFE film part. At the same time, the second stent 200 overlapped in several layers is rigid and thus may not be turned over in a direction opposite to the pulling direction of the first stent 100.

That is, when the anti-migration stent 1000 could not come out of the lumen 1, there was a risk that the lumen 1 would be damaged by the rigid second stent 200.

In order to solve this problem, the third stent 200 of the present disclosure has the third film part 230 made of silicone rather than PTFE to make the third film part 230 thin.

The anti-migration stent 1000 is manufactured by sewing and connecting the first space part 111 of one end of the first stent 100 and the second space part 211 of the connection part 220 to each other along a circumferential direction with the connecting thread 300 after the second stent 200 is fitted over one side of the first stent 100.

Here, one end of the first stent 100 and one end of the connection part 220 are located almost on the same line.

Accordingly, the procedure of the lesion area is performed by inserting the anti-migration stent 1000 into the lesion area 1a narrowed or blocked in the lumen 1 of the body, such as the esophagus, duodenum, and biliary tract of the human body, through a stent delivery system such as a catheter, and accordingly, as illustrated in FIG. 33, the first stent 100 expands the lesion area 1a occurring in the lumen 1 of the body, and the second stent 200 is inserted into and held in the lumen 1 in which the lesion area 1a has occurred.

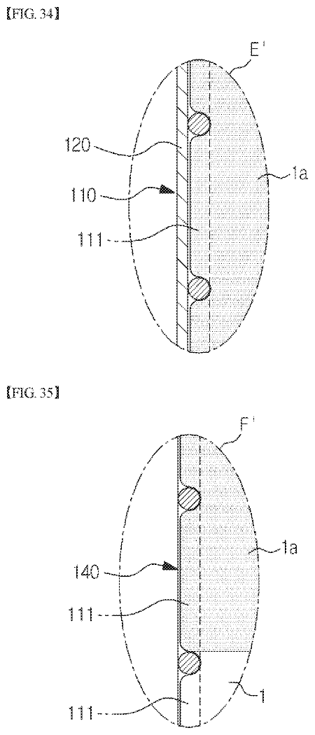

As illustrated in FIG. 34, the lesion area 1a is inserted into and held in the first space part 111 which is not closed by the first and second film parts 120, 130 bonded to each other, and the first film part 120 formed on the entire inner surface of the first cylindrical body 110 prevents the lesion area 1a from being inserted into the first cylindrical body 110 through the first space part 111. Of course, when the second stent 200 is located at the lesion area 1a, the third film part 230 prevents the lesion area 1a from being inserted into the second space part 211.

As illustrated in FIG. 35, the lumen 1 and the lesion area 1a are inserted into and held in the first space part 111 in the protruding part 140.

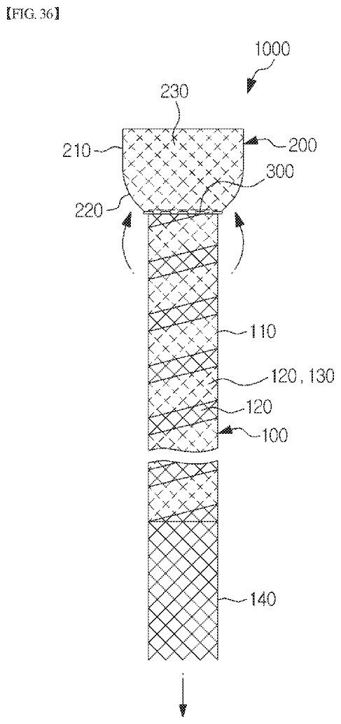

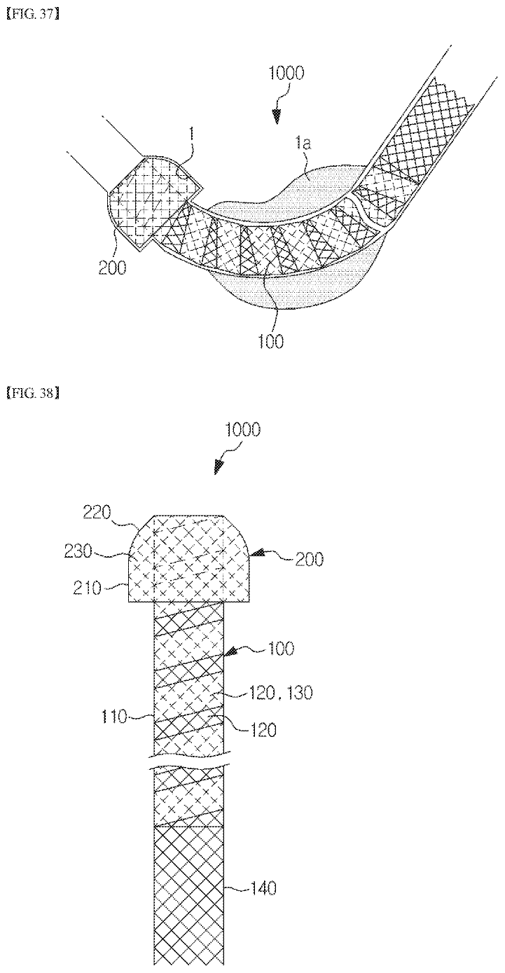

As illustrated in FIG. 36, when a predetermined period of time elapses after completing the procedure of the anti-migration stent 1000 on the lesion area 1a narrowed and blocked in the lumen 1 of the body, the anti-migration stent 1000 is required to be removed. In this case, the protruding part 140 of the first stent 100 of the anti-migration stent 1000 is pulled by the stent delivery system, such as a catheter.

Due to this action, the second stent 200 is turned over in a direction opposite to the pulling direction of the first stent 100 since the second stent 200 is in close contact with the lumen 1. Accordingly, the second stent 200 is not held in the lumen 1, and thus the anti-migration stent 1000 of the present disclosure can be easily removed from the lumen 1.

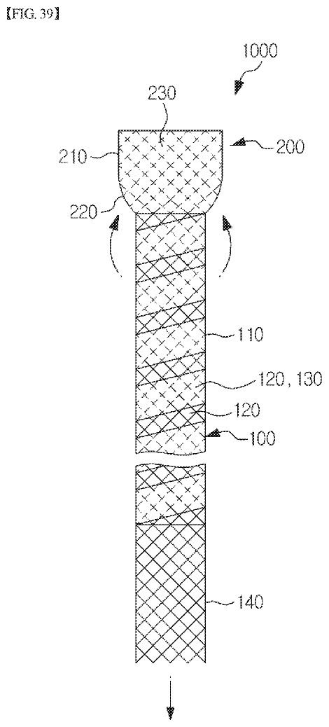

As illustrated in FIG. 37, when the anti-migration stent 1000 is located at the curved lumen 1, first space parts 111 are deformed in various parts of the first stent 100 flexible due to the free first film part 120, and this means that the anti-migration stent 1000 is easily deformed to fit the curved shape of the lumen 1.

In this case, the first space part 111 is not easily deformed in the remaining part of the first stent 100 which is not flexible due to the first and second film parts 120, 130 maintaining a bonded state. This means that unless the first space part 111 located at the free first film part 120 receives an external force, the first space part 111 maintains a deformed state due to the first space part 111 which is not easily deformed by being located at the first and second film parts 120, 130 maintaining a bonded state. That is, it means that the anti-migration stent 1000 maintains a curvedly deformed state.

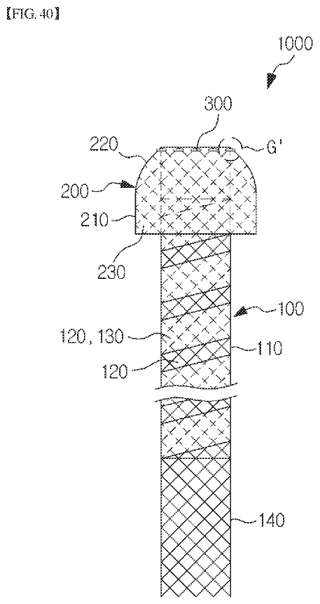

As illustrated in FIGS. 38 and 39, in the second stent 200 of the anti-migration stent 1000 according to the first modified example of the second embodiment of the present disclosure, without the connecting thread 300, the connection part 220 is integrally formed on and connected to one end of the first stent 100. When a predetermined period of time elapses after the procedure of the anti-migration stent 1000, the anti-migration stent 1000 is required to be removed. In this case, when the first stent 100 of the anti-migration stent 1000 is pulled, the second stent 200 is not held in the lumen 1 while being turned over in a direction opposite to the pulling direction of the first stent 100 since the second stent 200 is in close contact with the lumen 1, so the anti-migration stent 1000 is removed from the lumen 1.

In addition, since the connection part 220 does not have the connecting thread 300, the connection part 220 does not have a portion partially folded by the connecting thread 300.

That is, a connection portion of the connection part 220 connected to one end of the first stent 100 is prevented from protruding to the outer side of the first stent 100 while being turned over, so the anti-migration stent 1000 of the present disclosure is easily removed from the lumen.

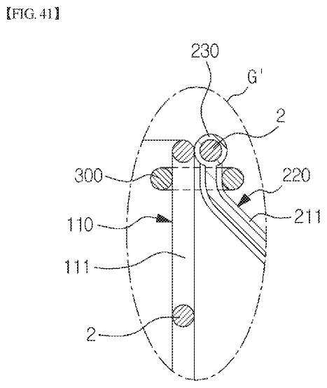

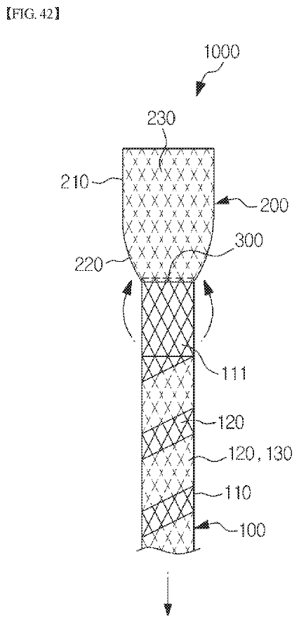

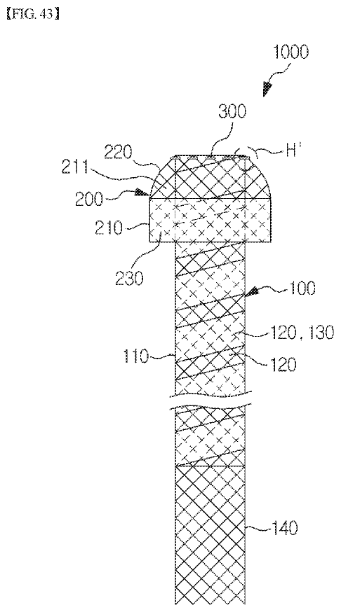

As illustrated in FIGS. 40 to 42, in the anti-migration stent 1000 according to the second modified example of the second embodiment of the present disclosure, one side of the first cylindrical body 110 overlapping with the connection part 220 has no first and second film parts 120, 130 formed thereon.

That is, the one side of the first cylindrical body 110 overlapping with the connection part 220 is flexible without first and second film parts 120, 130.

In addition, the connection portion of one end of the first stent 100 to the connection part 220 has no first and second film parts 120, 130 formed thereon and is configured to be thin.

According to such a configuration, when a predetermined period of time elapses after completing the procedure of the anti-migration stent 1000 on the lesion area 1a, the anti-migration stent 1000 is required to be removed. In this case, when the first stent 100 of the anti-migration stent 1000 is pulled, the second stent 200 is turned over in a direction opposite to the pulling direction of the first stent 100 since the second stent 200 is in close contact with the lumen 1, and one side of the first cylindrical body 110 flexible without the first and second film parts 120, 130 is easily deformed and stretched by an external force pulling the first stent 100, so the anti-migration stent 1000 of the present disclosure is easily removed from the lumen 1.

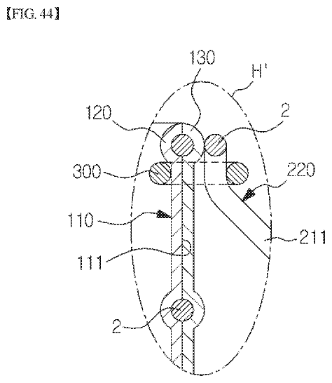

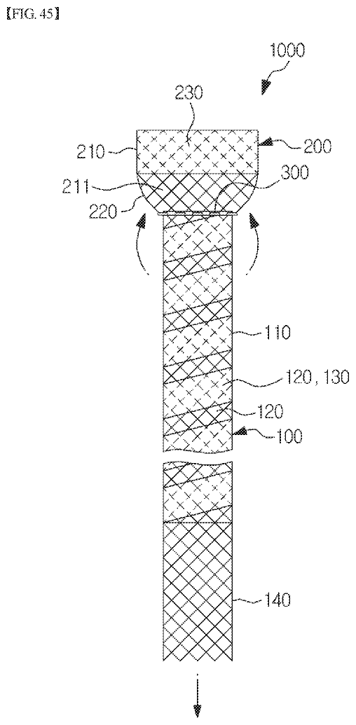

As illustrated in FIGS. 43 to 45, in the anti-migration stent 1000 according to the third modified example of the second embodiment of the present disclosure, the connection part 220 has no third film part 230 formed thereon.

That is, the connection part 220 is flexible without a third film part 230, and the connection portion of one end of the first stent 100 to the connection part 220 has no third film part 230 formed thereon and is configured to be thin.

According to such a configuration, when a predetermined period of time elapses after completing the procedure of the anti-migration stent 1000 on the lesion area 1a, the anti-migration stent 1000 is required to be removed. In this case, when the first stent 100 of the anti-migration stent 1000 is pulled, the second stent 200 is turned over in a direction opposite to the pulling direction the first stent 100 since the second stent 200 is in close contact with the lumen 1.

Here, due to the connection part 220 flexible without the third film part 230, the second stent 200 is easily deformed and turned over by an external force pulling the first stent 100.

For this reason, the anti-migration stent 1000 of the present disclosure is easily removed from the lumen 1.

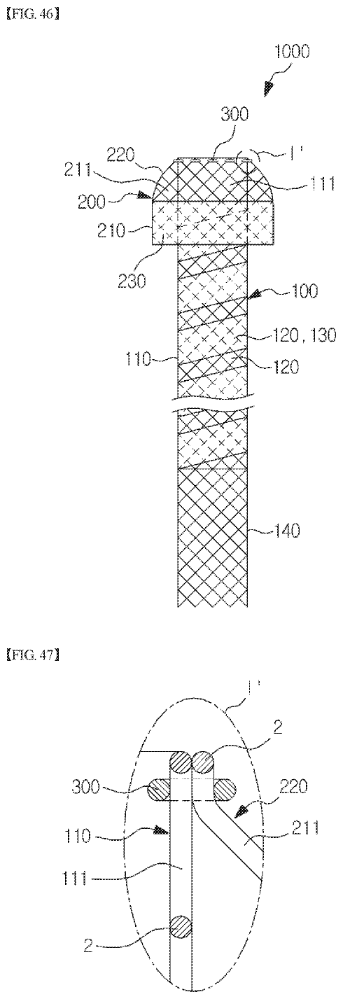

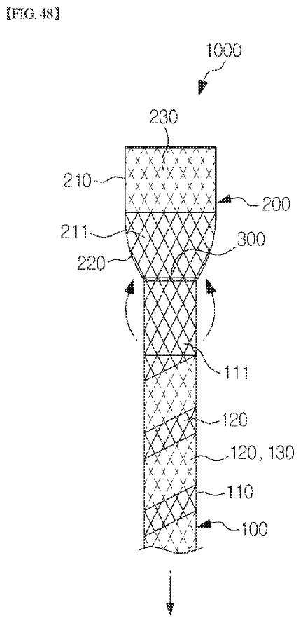

As illustrated in FIGS. 46 to 48, in the anti-migration stent 1000 according to the fourth modified example of the second embodiment of the present disclosure, one side of the first cylindrical body 110 overlapping with the connection part 220 has no first and second film parts 120, 130 formed thereon, and the connection part 220 has no third film part 230 formed thereon.

That is, the one side of the first cylindrical body 110 overlapping with the connection part 220 is flexible without first and second film parts 120, 130, and the connection part 220 is flexible without a third film part 230.

In addition, the connection portion of one end of the first stent 100 to the connection part 220 has no first, second, and third film parts 120, 130, 230 formed thereon and is configured to be thin.

According to such a configuration, when a predetermined period of time elapses after completing the procedure of the anti-migration stent 1000 on the lesion area 1a, the anti-migration stent 1000 is required to be removed. In this case, when the first stent 100 of the anti-migration stent 1000 is pulled, the second stent 200 is turned over in a direction opposite to the pulling direction of the first stent 100 since the second stent 200 is in close contact with the lumen 1.

Here, due to the connection part 220 flexible without the third film part 230, the second stent 200 is easily deformed and easily turned over by an external force pulling the first stent 100.

In addition, since one side of the first cylindrical body 110 flexible without the first and second film parts 120, 130 is easily deformed and stretched by an external force pulling the first stent 100, the anti-migration stent 1000 of the present disclosure is easily removed from the lumen 1.

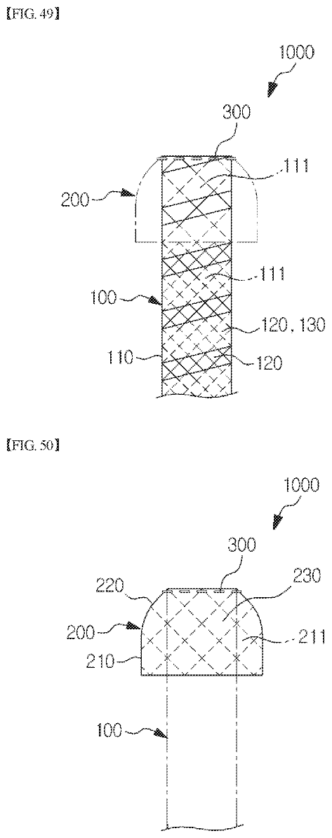

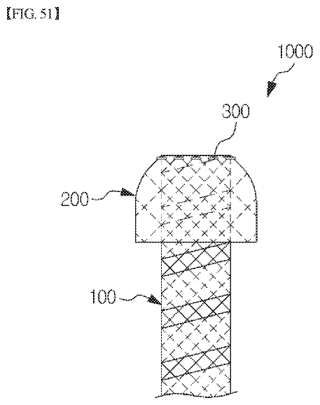

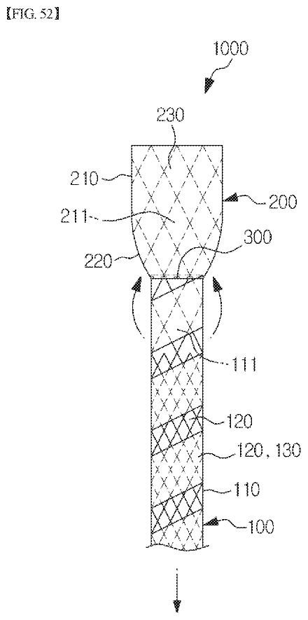

As illustrated in FIGS. 49 to 52, in the anti-migration stent 1000 according to the fifth modified example of the second embodiment of the present disclosure, a first space part 111 at one side of the first cylindrical body 110 overlapping with the second stent 200 is formed to be two to four times greater in size than a first space part 111 of a remaining part of the first cylindrical body 110 which does not overlap with the second stent 200.

That is, the one side of the first cylindrical body 110 overlapping with the second stent 200 has lower elasticity than the remaining part of the first cylindrical body 110 which does not overlap with the second stent 200.

According to such a configuration, when a predetermined period of time elapses after completing the procedure of the anti-migration stent 1000 on the lesion area 1a, the anti-migration stent 1000 is required to be removed. In this case, when the first stent 100 of the anti-migration stent 1000 is pulled, the one side of the first cylindrical body 110 overlapping with the second stent 200, due to low elasticity thereof, is easily deformed and stretched by an external force pulling the first stent 100.

In addition, since the second space part 211 of the second stent 200 is formed to be two to four times greater in size than the first space part 111 at the one side of the first cylindrical body 110 which does not overlap with the second stent 200, the elasticity of the second stent 200 decreases.

According to such a configuration, when a predetermined period of time elapses after completing the procedure of the anti-migration stent 1000 on the lesion area 1a, the anti-migration stent 1000 is required to be removed. In this case, when the first stent 100 of the anti-migration stent 1000 is pulled, the second stent 200 with low elasticity is easily deformed and easily turned over by an external force pulling the first stent 100, so the anti-migration stent 1000 of the present disclosure is easily removed from the lumen 1.

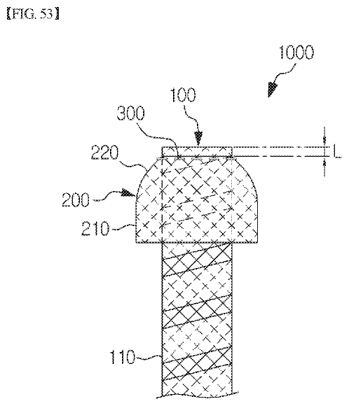

As illustrated in FIG. 53, the first stent 100 of the anti-migration stent 1000 according to the sixth modified example of the second embodiment of the present disclosure protrudes by a predetermined length L from the connection part 220 and is connected to the connection part 220 by sewing with the connecting thread 300. Here, since the length L is 3˜5 mm, one end of the first stent 100 and one end of the connection part 220 are not located on the same line.

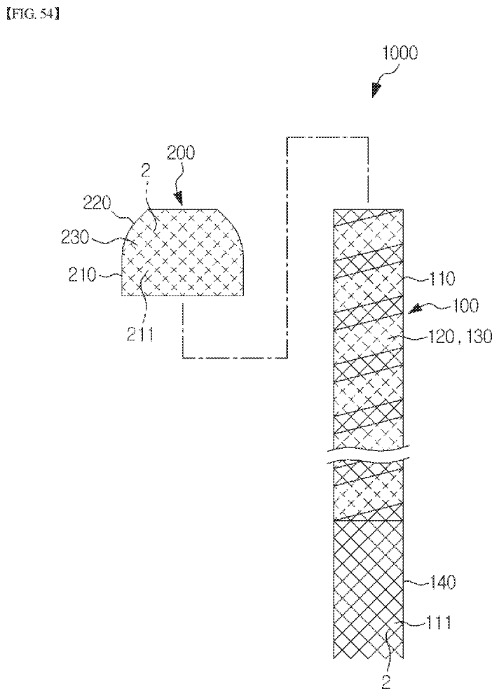

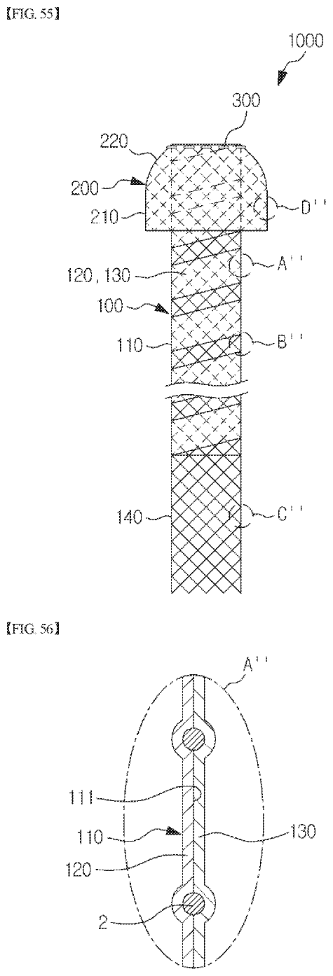

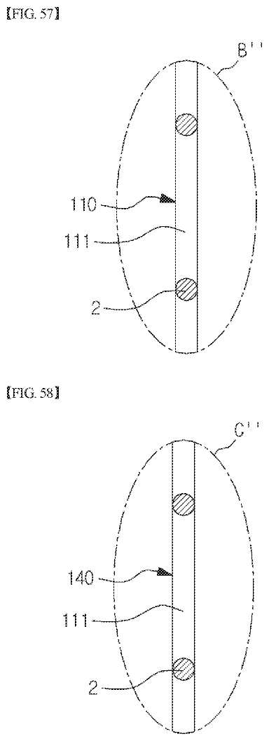

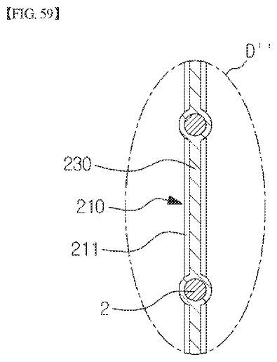

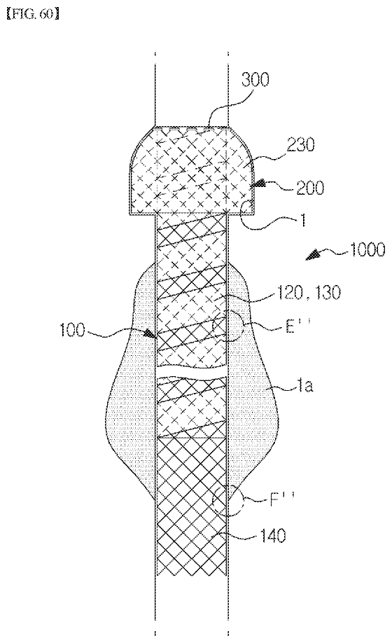

As illustrated in FIGS. 54 to 64, the anti-migration stent 1000 according to a third embodiment of the present disclosure includes: the first stent 100; the second stent 200 fitted over one outer side of the first stent 100 to be connected thereto by a connecting thread 300.

The first stent 100 includes the first cylindrical body 110 formed in a hollow cylindrical shape by weaving or crossing wires 2 made of a super elastic shape memory alloy in a mesh shape so that multiple first space parts 111 are formed between the wires 2.

Here, the first stent 100 is heat-treated, and a weaving part in which wires 2 are woven and a crossing part in which the wires 2 cross are formed around the first space part 111, and thus the first space part 111 is formed in a shape similar to a rhombus.

The first stent 100 includes the first film part 120 made of polytetrafluoroethylene (PTFE) formed on the inner surface of the first cylindrical body 110, with the first film part 120 formed in a spiral shape to have predetermined intervals defined therein; and the second film part 130 made of polytetrafluoroethylene (PTFE) formed on the outer surface of the first cylindrical body 110, with the second film part 130 formed in the same spiral shape as the shape of the first film part 120 to have predetermined intervals defined therein and bonded to the first film part 120.

Here, the first and second film parts 120, 130 are bonded to each other by heating and pressing, and some of the multiple first space parts 111 are closed by the first and second film parts 120, 130 bonded to each other.

That is, the first space part 111 closed by the first and second film parts 120, 130 is not easily deformed by an external force, and the first and second film parts 120, 130 are formed in spiral shapes on a portion of each of the inner and outer surfaces of the first cylindrical body 110, so the remaining portion of the inner and outer surfaces of the first cylindrical body 110 has no first and second film parts 120, 130 formed thereon.

For this reason, the remaining first space part 111 which is not closed by the first and second film parts 120, 130 may be deformed by an external force.

In other words, various parts of the first cylindrical body 110 are flexible with no first and second film parts 120, 130 formed thereon, and the remaining part of the first cylindrical body 110 is not flexible due to the first and second film parts 120, 130 maintaining a bonded state.

In addition, the intervals defined in the first film part 120 is regular or irregular, and the intervals defined in the second film part 130 are defined to be the same as the intervals of the first film part 120.

The first stent 100 includes the protruding part 140 formed to protrude on one side of the first cylindrical body 110 located at a side opposite to the second stent 200. At this time, the protruding part 140 has no first and second film parts 120, 130 formed thereon and thus the first space part 111 of the protruding part 140 is not closed.

The second stent 200 includes: the second cylindrical body 210 formed in a hollow cylindrical shape by weaving or crossing wires 2 made of a super elastic shape memory alloy are woven or crossed in a mesh shape so that multiple second space parts 211 are formed between the wires 2, wherein the second cylindrical body 210 is shorter in length than the first cylindrical body 110 and is a greater in diameter than the first cylindrical body 110; and the connection part 220 formed by having a diameter decreasing at one side of the second cylindrical body 210.

Here, the second stent 200 is heat-treated, and a weaving part in which wires 2 are woven and a crossing part in which the wires 2 cross are formed around the second space part 211, and thus the second space part 211 is formed in a shape similar to a rhombus. The second stent 200 includes the third film part 230 made of silicone formed on the second cylindrical body 210 and the connection part 220.

As for the third film part 230, multiple second space parts 211 are impregnated or spray-coated with a silicone solution, and each of the second space parts 211 is closed with the third film part 230 made of silicone.

To explain the reason for which the third film part 230, which is made of silicone rather than PTFE, is formed on the second stent 200, when the third film part is made of PTFE, one pair of third film parts is formed and is located to face each other on the inner and outer surfaces of the second stent 200, and then bonded to each other by heating and pressing, so the third film part made of PTFE thicker than the third film part 230 made of silicone is formed on the second stent 200.

Because of this, when the first stent 100 of the anti-migration stent 1000 is pulled to be removed when a predetermined period of time elapses after completing the procedure of the anti-migration stent 1000 on the lesion area 1a, the second stent 200 may be overlap in several layers by being rolled due to the thick PTFE film part. At the same time, the second stent 200 overlapped in several layers is rigid and thus may not be turned over in a direction opposite to the pulling direction of the first stent 100.

That is, when the anti-migration stent 1000 could not come out of the lumen 1, there was a risk that the lumen 1 would be damaged by the rigid second stent 200.

In order to solve this problem, the third stent 200 of the present disclosure has the third film part 230 made of silicone rather than PTFE to make the third film part 230 thin.

The anti-migration stent 1000 is manufactured by sewing and connecting the first space part 111 of one end of the first stent 100 and the second space part 211 of the connection part 220 to each other along a circumferential direction with the connecting thread 300 after the second stent 200 is fitted over one side of the first stent 100.

Here, one end of the first stent 100 and one end of the connection part 220 are located almost on the same line.

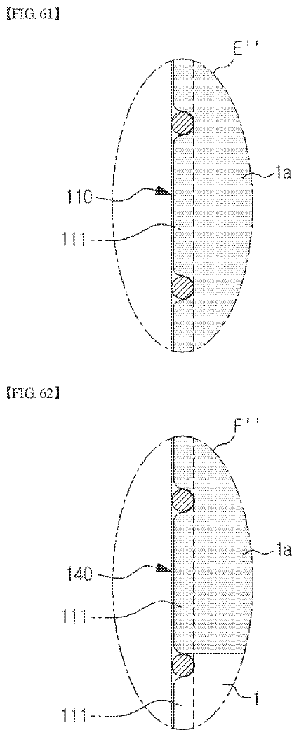

Accordingly, the procedure of the lesion area is performed by inserting the anti-migration stent 1000 into the lesion area 1a narrowed or blocked in the lumen 1 of the body, such as the esophagus, duodenum, and biliary tract of the human body, through a stent delivery system such as a catheter, and accordingly, as illustrated in FIG. 60, the first stent 100 expands the lesion area 1a occurring in the lumen 1 of the body, and the second stent 200 is inserted into and held in the lumen 1 in which the lesion area 1a has occurred.

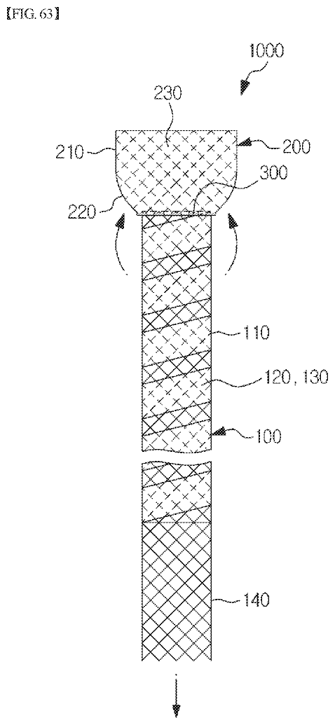

As illustrated in FIG. 61, the lesion area 1a inserted into and held in the first space part 111 which is not closed by the first and second film parts 120, 130 bonded to each other.

Here, when the second stent 200 is located at the lesion area 1a, the third film part 230 prevents the lesion area 1a from being inserted into the second space part 211.

As illustrated in FIG. 62, the lumen 1 and the lesion area 1a are inserted into and held in the first space part 111 in the protruding part 140.

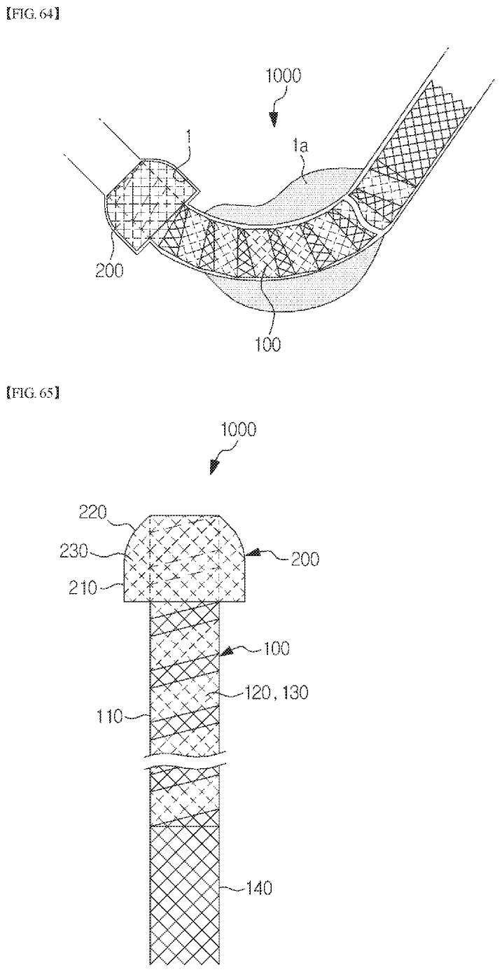

As illustrated in FIG. 63, when a predetermined period of time elapses after completing the procedure of the anti-migration stent 1000 on the lesion area 1a narrowed and blocked in the lumen 1 of the body, the anti-migration stent 1000 is required to be removed. In this case, the protruding part 140 of the first stent 100 of the anti-migration stent 1000 is pulled by the stent delivery system, such as a catheter.

Due to this action, the second stent 200 is turned over in a direction opposite to the pulling direction of the first stent 100 since the second stent 200 is in close contact with the lumen 1. Accordingly, the second stent 200 is not held in the lumen 1, and thus the anti-migration stent 1000 of the present disclosure can be easily removed from the lumen 1.

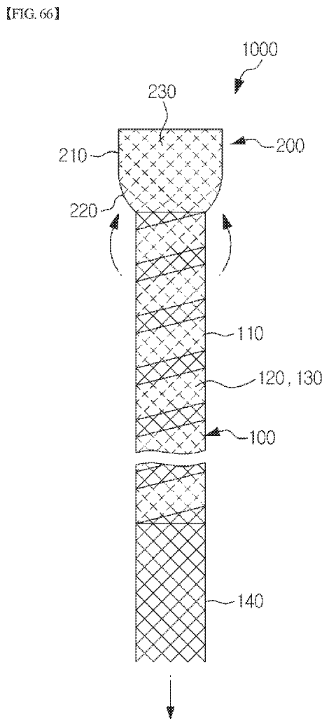

As illustrated in FIG. 64, when the anti-migration stent 1000 is located at the curved lumen 1, first space parts 111 are deformed in various parts of the first stent 100 flexible without the first and second film parts 120, 130, and this means that the anti-migration stent 1000 is easily deformed to fit the curved shape of the lumen 1.

In this case, the first space part 111 is not easily deformed in the remaining part of the first stent 100 which is not flexible due to the first and second film parts 120, 130 maintaining a bonded state. This means that unless the first space part 111 which is not located at the first and second film parts 120, 130 receives an external force, the first space part 111 maintains a deformed state due to the first space part 111 which is not easily deformed by being located at the first and second film parts 120, 130 maintaining a bonded state. This means that the anti-migration stent 1000 maintains a curvedly deformed state.

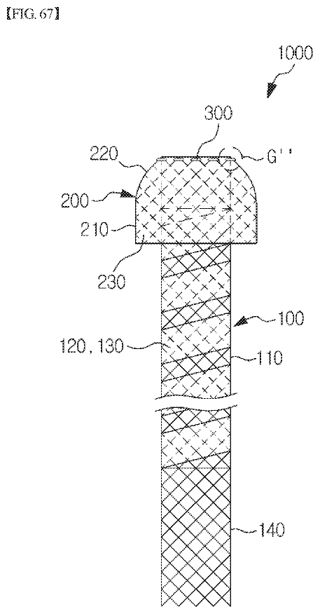

As illustrated in FIGS. 65 and 66, in the second stent 200 of the anti-migration stent 1000 according to the first modified example of the third embodiment of the present disclosure, without the connecting thread 300, the connection part 220 is integrally formed on and connected to one end of the first stent 100. When a predetermined period of time elapses after the procedure of the anti-migration stent 1000, there occurs a case in which the anti-migration stent 1000 is required to be removed. In this case, when the first stent 100 of the anti-migration stent 1000 is pulled, the second stent 200 is not held in the lumen 1 while being turned over in a direction opposite to the pulling direction of the first stent 100 since the second stent 200 is in close contact with the lumen 1, so the anti-migration stent 1000 is removed from the lumen 1.

In addition, since the connection part 220 does not have the connecting thread 300, the connection part 220 does not have a portion partially folded by the connecting thread 300.

That is, a connection portion of the connection part 220 connected to one end of the first stent 100 is prevented from protruding to the outer side of the first stent 100 while being turned over, so the anti-migration stent 1000 of the present disclosure is easily removed from the lumen.

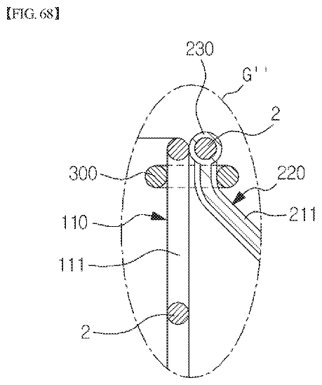

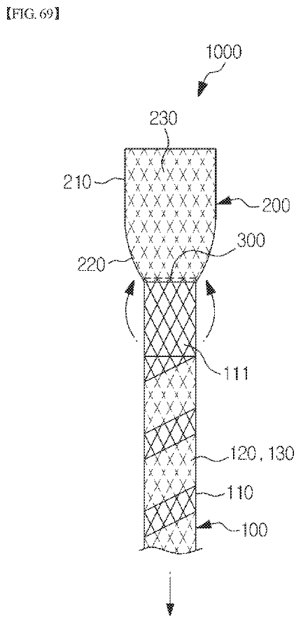

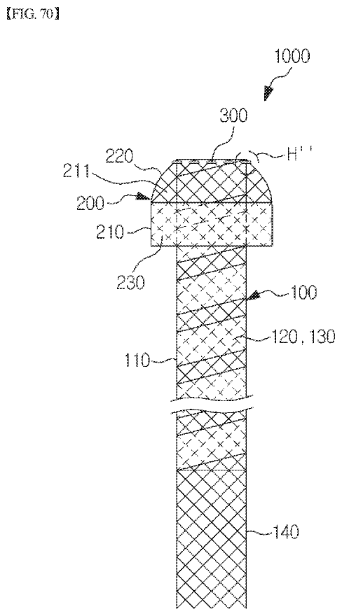

As illustrated in FIGS. 67 to 69, in the anti-migration stent 1000 according to the second modified example of the third embodiment of the present disclosure, one side of the first cylindrical body 110 overlapping with the connection part 220 has no first and second film parts 120, 130 formed thereon.

That is, the one side of the first cylindrical body 110 overlapping with the connection part 220 is flexible without first and second film parts 120, 130.

In addition, the connection portion of one end of the first stent 100 to the connection part 220 has no first and second film parts 120, 130 formed thereon and is configured to be thin.

According to such a configuration, when a predetermined period of time elapses after completing the procedure of the anti-migration stent 1000 on the lesion area 1a, the anti-migration stent 1000 is required to be removed. In this case, when the first stent 100 of the anti-migration stent 1000 is pulled, the second stent 200 is turned over in a direction opposite to the pulling direction of the first stent 100 since the second stent 200 is in close contact with the lumen 1, and one side of the first cylindrical body 110 flexible without the first and second film parts 120, 130 is easily deformed and stretched by an external force pulling the first stent 100, so the anti-migration stent 1000 of the present disclosure is easily removed from the lumen 1.

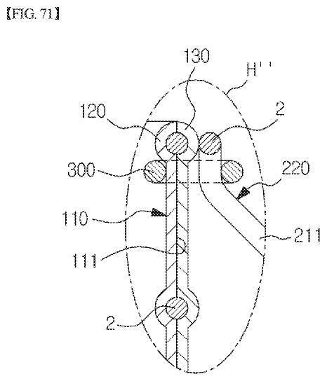

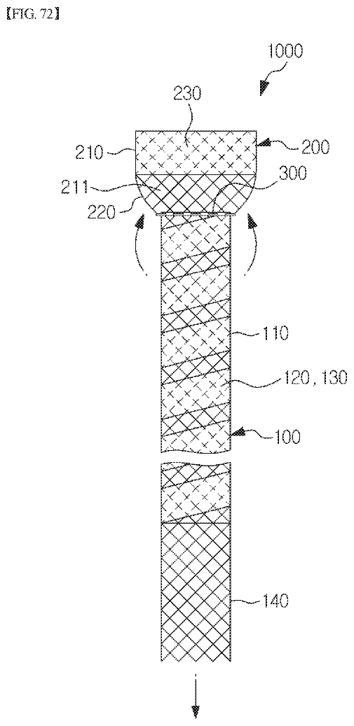

As illustrated in FIGS. 70 to 72, in the anti-migration stent 1000 according to the third modified example of the third embodiment of the present disclosure, the connection part 220 has no third film part 230 formed thereon.

That is, the connection part 220 is flexible without a third film part 230, and the connection portion of one end of the first stent 100 to the connection part 220 has no third film part 230 formed thereon and is configured to be thin.

According to such a configuration, when a predetermined period of time elapses after completing the procedure of the anti-migration stent 1000 on the lesion area 1a, the anti-migration stent 1000 is required to be removed. In this case, when the first stent 100 of the anti-migration stent 1000 is pulled, the second stent 200 is turned over in a direction opposite to the pulling direction the first stent 100 since the second stent 200 is in close contact with the lumen 1.

Here, due to the connection part 220 flexible without the third film part 230, the second stent 200 is easily deformed and turned over by an external force pulling the first stent 100.

For this reason, the anti-migration stent 1000 of the present disclosure is easily removed from the lumen 1.

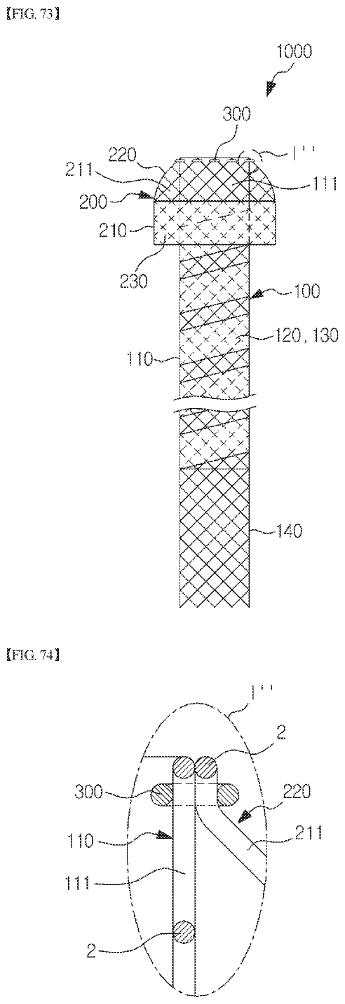

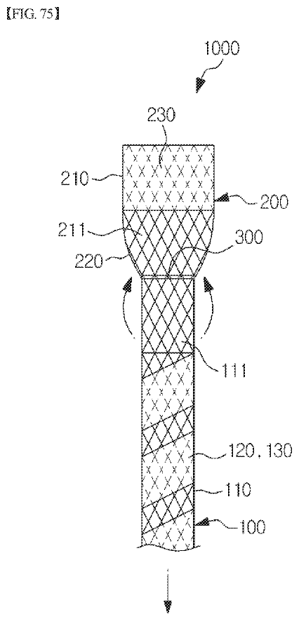

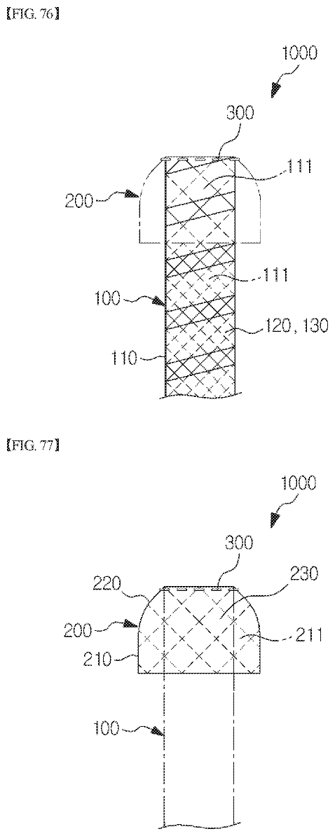

As illustrated in FIGS. 73 to 75, in the anti-migration stent 1000 according to the fourth modified example of the third embodiment of the present disclosure, one side of the first cylindrical body 110 overlapping with the connection part 220 has no first and second film parts 120, 130 formed thereon, and the connection part 220 has no third film part 230 formed thereon.

That is, the one side of the first cylindrical body 110 overlapping with the connection part 220 is flexible without first and second film parts 120, 130, and the connection part 220 is flexible without a third film part 230.

In addition, the connection portion of one end of the first stent 100 to the connection part 220 has no first, second, and third film parts 120, 130, 230 formed thereon and is configured to be thin.

According to such a configuration, when a predetermined period of time elapses after completing the procedure of the anti-migration stent 1000 on the lesion area 1a, the anti-migration stent 1000 is required to be removed. In this case, when the first stent 100 of the anti-migration stent 1000 is pulled, the second stent 200 is turned over in a direction opposite to a pulling direction of the first stent 100 since the second stent 200 is in close contact with the lumen 1.

Here, due to the connection part 220 flexible without the third film part 230, the second stent 200 is easily deformed and easily turned over by an external force pulling the first stent 100.

Since one side of the first cylindrical body 110 flexible without the first and second film parts 120, 130 is easily deformed and stretched by an external force pulling the first stent 100, the anti-migration stent 1000 of the present disclosure is easily removed from the lumen 1.

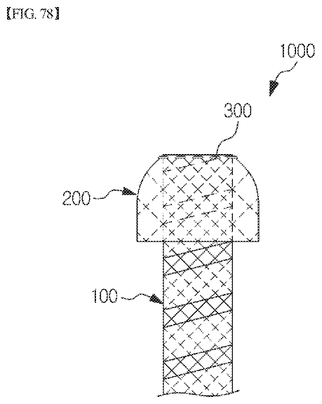

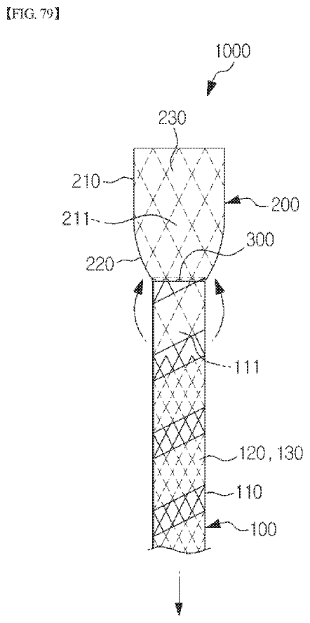

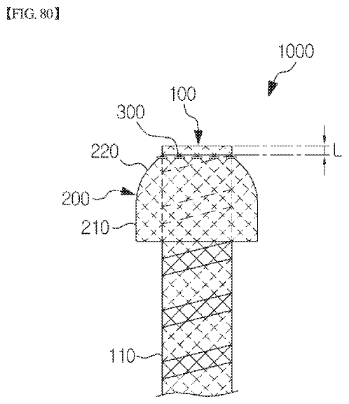

As illustrated in FIGS. 76 to 79, in the anti-migration stent 1000 according to the fifth modified example of the third embodiment of the present disclosure, a first space part 111 at one side of the first cylindrical body 110 overlapping with the second stent 200 is formed to be two to four times greater in size than a first space part 111 of a remaining part of the first cylindrical body 110 which does not overlap with the second stent 200.

That is, one side of the first cylindrical body 110 overlapping with the second stent 200 has lower elasticity than a remaining part of the first cylindrical body 110 which does not overlap with the second stent 200.

According to such a configuration, when a predetermined period of time elapses after completing the procedure of the anti-migration stent 1000 on the lesion area 1a, the anti-migration stent 1000 is required to be removed. In this case, when the first stent 100 of the anti-migration stent 1000 is pulled, the one side of the first cylindrical body 110 overlapping with the second stent 200, due to low elasticity, is easily deformed and stretched by an external force pulling the first stent 100.

Since the second space part 211 of the second stent 200 is formed to be two to four times greater in size than the first space part 111 at one side of the first cylindrical body 110 which does not overlap with the second stent 200, the elasticity of the second stent 200 decreases.

According to such a configuration, when a predetermined period of time elapses after completing the procedure of the anti-migration stent 1000 on the lesion area 1a, the anti-migration stent 1000 is required to be removed. In this case, when the first stent 100 of the anti-migration stent 1000 is pulled, the second stent 200, due to low elasticity, is easily deformed and turned over by an external force pulling the first stent 100, so the anti-migration stent 1000 of the present disclosure is easily removed from the lumen 1.

As illustrated in FIG. 80, the first stent 100 of the anti-migration stent 1000 according to the sixth modified example of the third embodiment of the present disclosure protrudes by a predetermined length L from the connection part 220 and is connected to the connection part 220 by sewing with the connecting thread 300. Here, since the length L is 3˜5 mm, one end of the first stent 100 and one end of the connection part 220 are not located on the same line.

In addition, when the second stent 200 of the anti-migration stent 1000 according to the various embodiments and modified examples of the present disclosure receives an external force horizontally in one direction within the lumen 1, the second stent 200 slants in a direction opposite to the receiving direction of the external force relative to the first stent 100.

That is, since the second stent 200 is strongly brought into close contact with and held in the lumen 1 while the second stent 200 moves by being slanted by an external force, the anti-migration stent 1000 does not migrate from the lesion area 1a.

In addition, when the second stent 200 receives an external force horizontally in both directions within the lumen 1, the second stent 200 protrudes in both directions intersecting with the receiving both directions of the external force relative to the first stent 100.

That is, since the second stent 200 is strongly brought into close contact with and held in the lumen 1 while being protruded by an external force, the anti-migration stent 1000 does not migrate from the lesion area 1a.

In the above, although the present disclosure has been illustrated and explained by taking specific preferred embodiments as examples, the present disclosure is not limited to the above-mentioned embodiments, and various changes and modifications may be made by those skilled in the art to which the present disclosure belongs within a scope without departing from the spirit of the present disclosure.

What is claimed is:

1. An anti-migration stent having a first stent (100) configured to expand a lesion area (1a) narrowed or blocked in a lumen (1) of a body and to have a first cylindrical body (110) formed in a hollow cylindrical shape by weaving or crossing wires (2) made of a super elastic shape memory alloy in a mesh shape so that multiple first space parts (111) are formed between the wires (2), a first film part (120) made of polytetrafluoroethylene (PTFE) formed on an inner surface of the first cylindrical body (110), with the first film part (120) formed in a spiral shape to have predetermined intervals defined therein, and a second film part (130) made of polytetrafluoroethylene (PTFE) formed on an entire outer surface of the first cylindrical body (110) so that the second film part (130) is bonded to the first film part (120), the anti-migration stent comprising:
a second stent (200) having multiple second space parts (211) formed by weaving or crossing the wires (2) made of a super elastic shape memory alloy in a mesh shape, with the second stent (200) being shorter in length than the first stent (100) and fitted over and connected to one side of the first stent (100) from the outside so that the second stent (200) is held in the lumen (1) in which the lesion area (1a) has occurred,
wherein the second stent (200) is composed of a second cylindrical body (210) formed in a hollow cylindrical shape, and a connection part (220) formed by having a diameter decreasing at one side of the second cylindrical body (210) so as to be connected to the first stent (100), with the second cylindrical body (210) and the connection part (220) having a third film part (230) made of silicone formed thereon.

2. The anti-migration stent of claim 1, wherein a first space part (111) of one end of the first stent (100) and a second space part (211) of the connection part (220) are sewn and connected to each other with a connecting thread (300).

3. The anti-migration stent of claim 1, wherein one end of the first stent (100) and the connection part (220) are integrally formed and connected to each other.

4. The anti-migration stent of claim 1, wherein a protruding part (140) is formed to protrude on one side of the first cylindrical body (110) located at a side opposite to the second stent (200),
wherein the protruding part (140) has no first and second film parts (120) (130) formed thereon.

5. The anti-migration stent of claim 1, wherein one side of the first cylindrical body (110) overlapping with the connection part (220) has no first and second film parts (120) (130) formed thereon.

6. The anti-migration stent of claim 1, wherein the connection part (220) has no third film part (230) formed thereon.

7. The anti-migration stent of claim 1, wherein one side of the first cylindrical body (110) overlapping with the connection part (220) has no first and second film parts (120) (130) formed thereon, and
the connection part (220) has no third film part (230) formed thereon.

8. The anti-migration stent of claim 1, wherein a first space part (111) at one side of the first cylindrical body (110) overlapping with the second stent (200) is greater in size than a first space part (111) of a remaining part of the first cylindrical body (110) which does not overlap with the second stent (200).

9. The anti-migration stent of claim 1, wherein each of the second space parts (211) is greater in size than each of the first space parts (111).

10. The anti-migration stent of claim 1, wherein the first stent (100) protrudes by a predetermined length L from the connection part (220) and is connected to the connection part (220).

11. An anti-migration stent having a first stent (100) configured to expand a lesion area (1a) narrowed and blocked in a lumen (1) of a body and to have a first cylindrical body (110) formed in a hollow cylindrical shape by weaving or crossing wires (2) made of a super elastic shape memory alloy in a mesh shape so that multiple first space parts (111) are formed between the wires (2), a first film part (120) made of polytetrafluoroethylene (PTFE) formed on an entire inner surface of the first cylindrical body (110), and a second film part (130) made of polytetrafluoroethylene (PTFE) formed on an outer surface of the first cylindrical body (110), with the second film part (130) formed in a spiral shape to have predetermined intervals defined therein and bonded to the first film part (120), the anti-migration stent comprising:
a second stent (200) having multiple second space parts (211) formed by weaving or crossing the wires (2) made of a super elastic shape memory alloy in a mesh shape, with the second stent (200) being shorter in length than the first stent (100) and fitted over and connected to one side of the first stent (100) from the outside so that the second stent (200) is held in the lumen (1) in which the lesion area (1a) has occurred,
wherein the second stent (200) is composed of a second cylindrical body (210) formed in a hollow cylindrical shape, and a connection part (220) formed by having a diameter decreasing at one side of the second cylindrical body 210 so as to be connected to the first stent (100), with the second cylindrical body (210) and the connection part (220) having a third film part 230 made of silicone formed thereon.

12. The anti-migration stent of claim 11, wherein a first space part (111) of one end of the first stent (100) and a second space part (211) of the connection part (220) are sewn and connected to each other with a connecting thread (300).

13. The anti-migration stent of claim 11, wherein one end of the first stent (100) and the connection part (220) are integrally formed and connected to each other.

14. The anti-migration stent of claim 11, wherein a protruding part (140) is formed to protrude on one side of the first cylindrical body (110) located at a side opposite to the second stent (200),
wherein the protruding part (140) has no first and second film parts (120) (130) formed thereon.

15. The anti-migration stent of claim 11, wherein one side of the first cylindrical body (110) overlapping with the connection part (220) has no first and second film parts (120) (130) formed thereon.

16. The anti-migration stent of claim 11, wherein the connection part (220) has no third film part (230) formed thereon.

17. The anti-migration stent of claim 11, wherein one side of the first cylindrical body (110) overlapping with the connection part (220) has no first and second film parts (120) (130) formed thereon, and
the connection part (220) has no third film part (230) formed thereon.

18. The anti-migration stent of claim 11, wherein a first space part (111) at one side of the first cylindrical body (110) overlapping with the second stent (200) is greater in size than a first space part (111) of a remaining part of the first cylindrical body (110) which does not overlap with the second stent (200).

19. The anti-migration stent of claim 11, wherein each of the second space parts (211) is greater in size than each of the first space parts (111).

20. The anti-migration stent of claim 11, wherein the first stent (100) protrudes by a predetermined length L from the connection part (220) and is connected to the connection part (220).

21. An anti-migration stent having
An anti-migration stent having a first stent (100) configured to expand a lesion area (1a) narrowed or blocked in a lumen (1) of a body and to have a first cylindrical body (110) formed in a hollow cylindrical shape by weaving or crossing wires (2) made of a super elastic shape memory alloy in a mesh shape so that multiple first space parts (111) are formed between the wires (2), a first film part (120) made of polytetrafluoroethylene (PTFE) formed on an inner surface of the first cylindrical body (110), with the first film part (120) formed in a spiral shape to have predetermined intervals defined therein, and a second film part (130) made of polytetrafluoroethylene (PTFE) formed on an outer surface of the first cylindrical body (110), with the second film part (130) being formed in the same spiral shape as the shape of the first film part (120) so as to have predetermined intervals defined therein and being bonded to the first film part (120), the anti-migration stent comprising:
a second stent (200) having multiple second space parts (211) formed by weaving or crossing the wires (2) made of a super elastic shape memory alloy in a mesh shape, with the second stent (200) being shorter in length than the first stent (100) and fitted over and connected to one side of the first stent (100) from the outside so that the second stent (200) is held in the lumen (1) in which the lesion area (1a) has occurred,
wherein the second stent (200) is composed of a second cylindrical body (210) formed in a hollow cylindrical shape, and a connection part (220) formed by having a diameter decreasing at one side of the second cylindrical body (210) so as to be connected to the first stent (100), with the second cylindrical body (210) and the connection part (220) having a third film part 230 made of silicone formed thereon.

22. The anti-migration stent of claim 21, wherein a first space part (111) of one end of the first stent (100) and a second space part (211) of the connection part (220) are sewn and connected to each other with a connecting thread (300).

23. The anti-migration stent of claim 21, wherein one end of the first stent (100) and the connection part (220) are integrally formed and connected to each other.

24. The anti-migration stent of claim 21, wherein a protruding part (140) is formed to protrude on one side of the first cylindrical body (110) located at a side opposite to the second stent (200),
wherein the protruding part (140) has no first and second film parts (120) (130) formed thereon.

25. The anti-migration stent of claim 21, wherein one side of the first cylindrical body (110) overlapping with the connection part (220) has no first and second film parts (120) (130) formed thereon.

26. The anti-migration stent of claim 21, wherein the connection part (220) has no third film part (230) formed thereon.

27. The anti-migration stent of claim 21, wherein one side of the first cylindrical body (110) overlapping with the connection part (220) has no first and second film parts (120) (130) formed thereon, and
the connection part (220) has no third film part (230) formed thereon.

28. The anti-migration stent of claim 21, wherein a first space part (111) at one side of the first cylindrical body (110) overlapping with the second stent (200) is greater in size than a first space part (111) of a remaining part of the first cylindrical body (110) which does not overlap with the second stent (200).

29. The anti-migration stent of claim 21, wherein each of the second space parts (211) is greater in size than each of the first space parts (111).

30. The anti-migration stent of claim 21, wherein the first stent (100) protrudes by a predetermined length L from the connection part (220) and is connected to the connection part (220).
